SEQUENCE LISTING (16 sequences)

SEQ ID NO: 1 - protein, 17 aa, Artificial Sequence
  Synthetic Peptide
KHHHKHHHKHHHKHHHK

SEQ ID NO: 2 - DNA, 21 bp, Artificial Sequence
  synthetic oligonucleotide
aauaauaauaauaauaauaau

SEQ ID NO: 3 - protein, 6 aa, Artificial Sequence
  Synthetic Peptide
HYFKFD

SEQ ID NO: 4 - protein, 6 aa, Artificial Sequence
  Synthetic Peptide
HFRRHL

SEQ ID NO: 5 - protein, 6 aa, Artificial Sequence
  Synthetic Peptide
HVHYYW

SEQ ID NO: 6 - protein, 6 aa, Artificial Sequence
  Synthetic Peptide
YYWLHH

SEQ ID NO: 7 - protein, 7 aa, Artificial Sequence
  Synthetic Peptide
HWRGWVC

SEQ ID NO: 8 - protein, 6 aa, Artificial Sequence
  Synthetic Peptide
HWRAWA

SEQ ID NO: 9 - protein, 6 aa, Artificial Sequence
  Synthetic Peptide
HWRGWA

SEQ ID NO: 10 - protein, 6 aa, Artificial Sequence
  Synthetic Peptide
HWRGWL

SEQ ID NO: 11 - protein, 6 aa, Artificial Sequence
  Synthetic Peptide
HWRAWV

SEQ ID NO: 12 - protein, 6 aa, Artificial Sequence
  Synthetic Peptide
HFRRHI

SEQ ID NO: 13 - protein, 6 aa, Artificial Sequence
  Synthetic Peptide
HFRRHA

SEQ ID NO: 14 - protein, 6 aa, Artificial Sequence
  Synthetic Peptide
HAHYYW

SEQ ID NO: 15 - protein, 6 aa, Artificial Sequence
  Synthetic Peptide
HWRGWV

SEQ ID NO: 16 - protein, 7 aa, Artificial Sequence
  Synthetic Peptide
HWRGWVA

(19) United States
(12) Patent Application Publication
(10) Pub. No.: US 2011/0312877 A1
(43) Pub. Date: Dec. 22, 2011

(54) ENGINEERED TUNABLE NANOPARTICLES FOR DELIVERY OF THERAPEUTICS, DIAGNOSTICS, AND EXPERIMENTAL COMPOUNDS AND RELATED COMPOSITIONS FOR THERAPEUTIC USE

(75) Inventors: Mark Berninger, Gaithersburg, MD (US); Puthupparampil Scaria, Montgomery Village, MD (US); Martin Woodle, Bethesda, MD (US)

(73) Assignee: APARNA BIOSCEIENCES, Rockville, MD (US)

(21) Appl. No.: 12/919,703

(22) PCT Filed: Feb. 26, 2009
(86) PCT No.: PCT/US09/35360

§ 371 Date: May 4, 2011

Related U.S. Application Data
(60) Provisional application No. 61/067,037, filed on Feb. 26, 2008.
(60) Provisional application No. 61/067,039, filed on Feb. 26, 2008.
(60) Provisional application No. 61/128,409, filed on May 22, 2008.
(60) Provisional application No. 61/136,750, filed on Sep. 30, 2008.

(51) Int. Cl. A61K 38/16; A61P 31/10; C07K 19/00; B82Y 5/00

(52) U.S. Cl. 514/3.3; 530/409; 530/387.3; 530/395; 530/399; 530/358; 530/352; 977/773; 977/795; 977/906; 977/927

(57) ABSTRACT

Biomedical nanoparticles are disclosed based on new engineered modular carrier macromolecules, on engineered macromolecules or associated entities providing an internal nanoparticle structure, and compositions for minimizing non-specific binding of the nanoparticles while enabling efficient and convenient targeting to cells and tissues. These nanoparticles may be used to deliver atomic or molecular or associated entities which are useful for diagnostics, primarily in vivo imaging, for therapeutics, for vaccines, or for experimental research. Nanoparticles comprising combinations of active entities such as gene inhibitors with gene expression cassettes or imaging agents with therapeutic agents, and polyamide compounds useful for treatment of microbial infections are also disclosed.

This application claims the benefit of U.S. Provisional Applications: 61/067,037, filed Feb. 26, 2008; 61/067,039 filed Feb. 26, 2008; 61/128,409 filed May 22, 2008, and 61/136,750, the contents of which are hereby incorporated by reference in their entireties.

### FIELD OF THE INVENTION

The invention relates to engineered macromolecules and nanoparticles for cell and tissue delivery of experimental, diagnostic, or therapeutic molecules including immunogens useful as vaccines, particularly compounds that are charged, and more particularly nucleic acids, peptides and anionic agents, and more particularly for delivery of these compounds in living animals or humans. The invention relates to nanoparticles useful for biomedical research, diagnosis, treatment or monitoring of treatment, or prevention of disease including by immunization.

### BACKGROUND OF THE INVENTION

Pharmaceutical based prevention and treatment of disease, diagnostic imaging and many types of biomedical research require that specific molecular entities be delivered to particular sites in living organisms. The molecular character of each of these agents is constrained and largely determined by its function as a therapeutic, a diagnostic imaging agent, or the experimental question it is intended to address. Modification of its molecular structure to optimize its delivery may, therefore, be impossible, or disadvantageous. Thus, whole classes of molecules, such as phosphorylated and other anionic agents, are often excluded from consideration for biomedical applications. Compositions and methods that can deliver a wide variety of molecular entities are highly desired. Further, the therapeutic, diagnostic, or experiment entity will be most effective if it is delivered preferentially to specific sites in the body. For instance, if the mode of action of a drug is to kill tumor cells, then preferential delivery to tumor cells will be desired, likewise for an imaging agent used to detect a pathological tissue. Biomedical research and the treatment and diagnosis of disease in humans will be advanced by improved methods and reagents for the delivery of compounds that affect the metabolism of specific cells and tissues in an experimental animal or a human patient. In the former case these improvements will accelerate biomedical research into causes of disease, diagnostic methods, and treatments. In the latter case the value is clear. In particular, limitations with respect to delivery of charged molecules including nucleic acids such as siRNA, has impeded research and been an obstacle to the development of therapeutics. Targeted charged imaging agents will improve diagnosis and monitoring of treatment. Also the range of molecules that are drug candidates has been restricted absent pharmaceutical delivery systems.

A number of nanometer scale delivery particles have been developed including liposome and albumin based particles capable of delivering such drugs as doxorubicin and TAXOL®. Liposomes entrap their cargo within an interior aqueous compartment. Other nanoparticle compositions have been developed in which the cargo is entrapped in a solid phase or bind to carrier components forming a solid phase. Many nanoparticles have been described based on synthetic polymers such as water insoluble polylactic-glycolic acid (PLGA) or positively charged polymers like polyethyleneimine (PEI) and co-polymers comprised of histidine and lysine monomers. Nanoparticle formulations have been described in which a carrier associates with an experimental or therapeutic or imaging compound, and may to varying degrees contribute to the particle structure. Such an experimental or therapeutic or imaging compound is hereinafter referred to as a cargo. The objective of many efforts to develop such nanoparticle systems has been to enable use of charged or water insoluble molecules as cargo for biomedical applications, where their charge or solubility has prevented such uses. For example, many nucleic acids could modulate gene activity if they can gain access to the interior of the cell in a pharmacologically acceptable manner, but more effective means to accomplish this are still needed. Many drugs given systemically, including many drugs for the treatment of cancer cause significant adverse side effects that reduce the quality of life of patients under treatment and limit the duration and dosage. These drugs generally exhibit little or no targeting to the cells or tissues where they provide therapeutic benefit. If they can be targeted to these cells and tissues relative to their now untargeted distribution their side effects could be reduced with significant benefit for patients in terms of quality of life and treatment of their disease. Delivery of drugs with a targeted nanoparticle has the potential to achieve this medical benefit. Insofar as cargos may vary in terms of their size and chemical properties it is desirable to have a variety of carrier macromolecules available to which cargos may be matched. This process of matching cargo compounds to carriers is hereinafter referred to as tuning the nanoparticle.

Among the desired properties of carrier molecules, in addition to their ability to form nanoparticle in association with a cargo, are, minimal toxicity, biodegradability, and ease and low cost of manufacture.

A major need exists for new or improved vaccines including vaccines for cancer treatment and prophylactic vaccines and treatments for infectious organisms, for instance anthrax, drug resistant bacteria and fungi, and viruses. Vaccines that can be developed, tested, and manufactured rapidly are especially valuable to address emerging organisms and organisms that may be developed by or disseminated by terrorist groups. Therapeutic vaccination promises to extend life span and improve the quality of life of patients afflicted with disease. In addition, improved therapeutics for treatment of fungal infections, which are often life threatening, are needed. This need is especially acute with respect to infections caused by microbial strains that already are resistant to currently available antifungal drugs or will become resistant.

Advances in molecular biology and recent discoveries concerning control of gene expression including RNA interference, antisense nucleic acid, gene based therapy, aptamers, and other novel compounds provide powerful tools for investigation of fundamental biological processes and the causes and mechanism or disease, and they further represent promising new therapeutic drugs to treat disease. Their use in research and in the clinic is limited by deficiencies in the currently available methods and reagents to enable their delivery to cells in an animal or a human, especially with respect to deficiencies in the ability of current methods and reagents targeted to specific cells or tissues in the body.

In addition to the aforementioned nanoparticles carriers and cargo it is desirable to coat the nanoparticle with components that stabilize the nanoparticle and reduce non-specific interactions and/or immunogenicity following exposure to cells or injection into an animal or human and to provide selective binding or interaction. Hereinafter hydrophilic macromolecular surface components that provide low non-specific binding and reduced immunogenicity are called steric coating. The steric coating also can be used to provide sites for attachment of ligands or similar binding moieties that may directly or indirectly provide means for specific targeting of the nanoparticle to cells that contain particular structures or receptors on their surface. Binding of the steric molecules has been carried out by conjugation with carrier either prior to nanoparticle formation or chemical reaction with carrier exposed on the nanoparticle surface. Improved methods to secure steric coating molecules to the surface of the nanoparticle are needed.

It has been possible to decorate nanoparticles with various ligands and binding groups displayed on the surface of the nanoparticle. Numerous agents have been used as targeting ligands. One important broad class of targeting ligands is peptides and another is antibodies. Antibodies have been used by conjugation to the antibody but this approach has many problems, including adverse effects on antibody function from the need to modify it, frequent use of random modification that adversely affects biological activity is not a defined product, makes reproducibility difficult or impossible, and exposes different parts of the antibody giving a multitude of biological activities from each exposed part. Random conjugation also fails to avoid the antigen binding region of the antibody. In order to achieve targeting with antibody ligands, attachment in a consistent and oriented fashion, without chemical modification of the antibody is needed. Control over the number of such ligands or binding groups per particle and the variation of number of such ligands or binding groups among nanoparticles in a population has need of improvement and is desired. It is an object of the invention to provide nanoparticles with improved control of ligand display on the surface.

One of the major challenges to development of nanoparticles has been identifying means to obtain control of the nanoparticle structure, size distribution, and colloidal stability. Obtaining polymer-based nanoparticles with a low polydispersion, and in particular a controlled average particle size remains a largely unmet need. The most common approach to solve this problem has been conjugation of a hydrophilic PEG polymer to the nanoparticle forming polymer, the drug carrier, so that a steric surface barrier is formed on the nanoparticle surface that reduces particle size growth during formation and reduces aggregation by providing a hydrophilic coating. Nonetheless, the use of conjugation between the PEG polymer and the nanoparticle forming polymer still present problems, including interference by the PEG with the nanoparticle formation and stability and retention of molecular heterogeneity that carriers over to the nanoparticle, and thus this method is one that nears improvement. Recently, an alternative form of electrostatically assembled nanoparticles for nucleic acids was developed using a homogenously cationic polypeptide comprising histidine and lysine copolymers to form association nanoparticles. Improved control of the nanoparticle structure and size distribution is needed. Thus there is a substantial need for better means to control nanoparticle structure, size distribution and colloidal stability.

Many microbial infections, such as fungal infections, represent a life threatening disease condition, usually manifested in immunocompromised patients, and for such patients treatment options are limited and entail undesired side effects. When the infecting microbe is resistant to currently used drugs treatment options are especially limited. A need exists for drugs that will expand treatment options, especially with respect to invasive fungal infectious that are resistant to currently available drugs. Certain engineered macromolecules used to form the above mentioned nanoparticles exhibit anti-microbial activity in their own right, that is, absent any separate therapeutic cargo. While these carrier compounds show promise as novel drugs their synthesis is difficult in many instances as well as costly, and this is an obstacle to their development and use as anti-microbial agents.

Lentiviral vectors have been used to delivery genes and other transcribable sequences to living cells in vitro and in vivo and, they hold promise as gene based drugs. Currently these vectors require the presence of an envelope protein, with this requirement usually met by the use of the VSG-G envelope protein, (Vesicular Stomatitis Virus G protein). Use of this envelope protein entails difficulties with respect to viral vector product and product safety. The use of a synthetic component such as those employed in the formulation of fully synthetic nanoparticles will obviate the need to use this viral component and will advance the development and use of lentiviral and similar vectors as therapeutics.

Nanoparticle formulations have been commercialized for therapeutics, e.g., DOXIL® and ABRAXANE®, others for vaccines, and yet others described in numerous studies for imaging agents, but these nanoparticles have not fully addressed the needs of peptides and other macromolecules, ionic agents, etc. for many biomedical applications. In particular, gene expression cassettes and siRNA gene inhibitors lack appropriate delivery means for experimental and clinical applications. Additionally, a particular challenge is a need to deliver combinations of active agents, such as an agent for expression of one gene in concert with an agent for inhibition of another gene, or a combination of imaging agents with therapeutic agents.

### SUMMARY OF THE INVENTION

The technology described herein provides improved macromolecules useful for antimicrobial activity and separately useful for the formation of nanoparticles, including polyamides comprising natural and non-natural amino acids. Use of such polyamides with versatile amino acid components expands the repertoire of carriers available to match particular cargo molecules, and the selection of carrier for each cargo constitutes tuning the nanoparticle to optimize performance. In one embodiment the technology described herein provides carriers matched for specific cargo molecules to obtain a stable association of the cargo molecule(s) within the nanoparticle while also facilitating tissue or intracellular release of the cargo. In one embodiment the invention provides a carrier that associates with cargo to form a nanoparticle comprising the cargo and in another embodiment the invention provides two carriers that associate with each other, as well as cargo, to form a nanoparticle comprising the cargo.

The invention provides non-natural amino acids as building blocks of polyamide carriers that greatly expand the range of biochemical properties of the carriers and the nanoparticles they form, thus providing 1) more effective intracellular delivery of macromolecular and/or ionic cargo and 2) means to tune the nanoparticle for a wide range of cargo not previously possible. Modular polyamide macromolecules, optionally branched, are provided comprising natural and non-natural amino acids with pendant organic nitrogen and/or oxygen containing groups, including but not limited to lysine, ornithine, diaminobutyrate, histidine, 2-methyl histidine, imidazole derivatives of diaminobutyrate, glutamate, aspartate, glutamine, asparagine, serine, tyrosine, and aminoglucuronate. Said polyamide macromolecules optionally comprise other activity pendent groups such as thiol or hydrophobic or aromatic moieties that associate with cargo, or with another polyamide carrier, or with surface coating material. Branched macromolecules are provided with 3 to 16 macromolecules as arms attached to a central core moiety comprising multiple attachment moieties (e.g., 3 to 25, 4 to 20, or 5 to 15 etc.) such as a JEFFAMINE®, amine surface poly amido amide (PAMAM) dendrimer of 1, 2 or 3 generations, branched PEI with a molecular weight less than 5 KID, ethylene diamine tetraacetic acid (EDTA), or a linear or circular polyamide with pendant linkage moieties, or a large number of arms may be attached to a large solid core with many attachment sites on its surface such as thiol binding to the surface of colloidal gold. Each arm may comprise from about 6 to about 50 amino acids, or from about 10 to about 40 amino acids, or from about 15 to about 30, or from about 12 to about 25 amino acids. Arms optionally comprise one to three branches within 30 to 100% of the arms.

Macro molecules arms may include branching species that provide sites for branching of the arms include, but are not limited to, neutral compact linkages such as lysine or glutamate or cysteine disulfide, flexible linkage such as with PEG or an aliphatic chain, charged branching species such as with carboxyspermine or carboxyspermidine, and/or reversible linkage such as reducible or acid cleavable. Macromolecule arms may also have a defined sequence, including but not limited to, repeating sequences, a unique sequence, or may have a random sequence and may have a structure that is either defined such as that produced by solid phase step wise synthesis or a polydisperse structure such as that produced by solution phase polymerization. The present invention provides methods for the synthesis of the aforementioned polyamide carriers.

Optionally 2 to 6 cores may be coupled together using flexible or rigid linkers such as PEG or branched DNA.

It has been possible to decorate nanoparticles with protective polymers and ligands displayed on the surface. However, control over the attachment of such polymers as well as number of such ligands has need of improvement and is desired. Note that association of a steric coating and/or targeting ligand directly with cargo is disadvantageous, even if by reversible covalent bonds, and a substantial advantage is obtained if modification of cargo is not required. In one embodiment nanoparticles are with improved control of protective polymers and ligands are provided. The technology described herein provides for an easy generation of antibody-targeted nanoparticles. Antibodies are a class of molecules that can be used as ligands to target specific cell types and tissue. They can be used to for targeted delivery of nanoparticles to specific cells. Their use for targeting nanoparticles is limited because of the need to have covalent conjugation of antibody molecules to the nanoparticle surface. This type of conjugation suffers from random modification of the antibody molecule, their random orientation on the nanoparticle surface, loss of biological activity due to chemical modification of binding sites, and variability in the product. The invention provides for a solution to these problems by the attachment of the antibody molecules to the nanoparticle surface through an antibody-binding molecule that binds through non-covalent interaction. The antibody-binding molecule binds to a specific site on the molecule providing uniform orientation of the bound antibody molecule. The antibody-binding molecule is attached to the nanoparticle surface directly or through a steric polymer. This allows easy replacement of the antibody depending on the target site and application, and also use of combination of antibodies.

The technology described herein also provides for synthesis of ligand-PEG-polycation conjugates where free amine of the ligand and the amino groups of polycation are coupled using a heterobifunctional PEG, SCM-PEG-Mal. The maleimide (Mal) moiety is known to be a sulfhydryl coupling group and hetero-bifunctional coupling agents such as SCM-PEG-Mal are commonly used to couple an amino group of one molecule to the sulfhydryl group of another molecule. Some embodiments provide for the use of this hetero-bifunctional molecule for coupling two amino compounds.

Conjugates are provided combining either a carrier-like chemical domain or alternatively a cargo-like chemical domain coupled to a hydrophilic domain such as a steric polymer such as polyethylene glycol (PEG), a polyanionic domain such as polyglutarnic acid (PGA), or an unstructured recombinant polymer such as described in US Patent 2008/0039341 A1. Such conjugates optionally also containing a ligand or linker preferably attached opposite the carrier-like or cargo-like domain. When a cargo-like domain is used, the hydrophilic coating or ligand preferably is conjugated to a biologically inactive chemical domain that closely resembles or is essentially chemically equivalent to chemical domains of the cargo, i.e. a pseudo-cargo. For example, hydrophilic coating optionally is provided via conjugation to macromolecules resembling cargo, such as DNA oligonucleotides bearing 10 to 50 negatively charged phosphate groups or short oligomers of glutamate bearing 10 to 50 pendant anionic carboxyls, either one resembling siRNA or a gene cassette. When a carrier-like domain is used, the hydrophilic coating or ligand preferably is conjugated to a single site, optionally at one end, such as to the carboxyl end of a short oligomer of orthinine bearing 10 to 50 pendant amines and thus resembling a branched macromolecule polyamide carrier bearing numerous imidazole and primary amine pendant groups. The resulting conjugate associates with the nanoparticle by binding of its carrier-like or cargo-like domain within the nanoparticle in such a manner that the hydrophilic domain coats the surface of the particle. The optionally included ligand or linker that is conjugated to the carrier-like domain is thereby is displayed on the outside surface of the nanoparticle where it exhibits its desired biochemical activity, e.g., binding to a cell surface receptor or attachment of a ligand such as an antibody or peptide ligand for a receptor. Linker moieties are provided such as hydrazide moieties to couple a ligand via an aldehyde or peptides that bind a ligand such as to an antibody Fc region thereby displaying and orienting the bound ligand to enable its specific binding target and thereby provide targeting to cells that contain a corresponding target.

In another embodiment, the technology described herein provides coating via hydrophilic macromolecules that comprise multiple attachment moieties, such as pendant aldehyde or hydrazide moieties on oxidized dextran that form a Schiff base with surface exposed carrier amines, forming a stabilizing and/or protective surface layer that is shed in the acidic endosome compartment. Said hydrophilic coating optionally further comprises hydrophilic material and optionally further comprises ligand or linker moieties.

In some embodiments, the present technology provides methods for the use of the aforementioned nanoparticles to deliver immunogenic molecules, immunostimulatory molecules, biologically active molecules for research or therapeutics, or agents that enable diagnostic imaging using methods including but not limited to radiation, magnetic resonance, positron emission tomography, ultrasound or other imaging techniques. The invention provides macromolecules with antimicrobial activity, including antifungal activity, or for treating wounds. The above compositions and methods may be used to develop new therapies or may themselves be used for ex-vivo or in vivo treatment or prevention primarily through vaccination to prevent or treat diseases including but not limited to cancer, diseases of the circulation, inflammatory disease, infectious disease, autoimmune disease, and neurological disease.

### BRIEF DESCRIPTION OF THE DRAWINGS

FIG. 1 shows an antibody binding to Fc binding peptide-PEG-PEI conjugate as a function of antibody concentration. Argarose gel experiment showed that the PPP/DNA/Ab NPXs remained intact after purification. PFP/DNA/IgG NPXs were purified from free antibody and PPP by Sephacryl S-500-HR microspin column. Lane 1: pcDNA-Luc plasmid 0.5 ug; Lane 2: unpurified PPP/DNA/IgG NPXs; Lane 3: unpurified PPP/DNA/IgG NPXs preincubated with 0.7 mg/ml of heparin; Lane 4: purified PPP/DNA/IgG NPXs; Lane 5: purified PPP/DNA/IgG NPXs preincubated with 0.7 mg/ml of heparin (A). Dot blotting assay. Human IgG (400 mg/ml) and PPP/DNA/IgG nanoparticles (containing 400 mg/ml IgG) were purified from free antibody and PPP by Sephacryl S-500-HR microspin column. The purified and unprified antibody and NPXs was spot into a nitrocellular membrane. Anti-human IgG-HRP was used to detect antibody concentration of purified and unpurified antibody and NPXs. Lane 1&2: background; Lane 3. human IgG (before purification); Lane 4. human IgG (after purification); Lane 5. PPP/DNA/IgG NPXs (before purification). Lane 6. PPP/DNA/IgG NPXs (after purification) (B). Peptides were added to PPP prior to addition of antibody, except triangle data points where peptide 0 was added after antibody, lowering competition due to a long off-rate for antibody dissociation from PPP (C).

FIG. 2 shows a depiction of modular branched polyamide macromolecules comprising pendant groups and combinatorial components for preparing such compositions.

FIG. 3. Depicts diaminocarboxylate compounds (DAC) and their organic nitrogen pendant groups.

FIG. 4 shows some repeating units and arms comprising organic nitrogen pendant groups.

FIG. 5 shows example of compositions comprising cationic core-flexible spacer-trimer with 2-cationic branch macromolecule comprising pendant organic nitrogen groups plus aliphatic end groups and PEG-ligand (5A). An example of a neutral core-dimer, 4-high cationic branch macromolecule comprising pendant organic nitrogen groups ±PEG-ligand (5B). An example of a neutral core-dimer, 4-low cationic branch macromolecule comprising pendant organic nitrogen groups and aliphatic end groups (5C).

FIG. 6 depicts an example of a nanoparticle forming macromolecule composition for packaging nucleic acid comprising a cationic core-dimer with PEG-peptide ligand conjugate, 4-high cationic branch macromolecule comprising cationic pendant organic nitrogen groups and cationic end groups (6A). An example of an antibody binding macromolecule composition comprising a neutral core-dimer with PEG-peptide conjugate, 4-low cationic branch macromolecule comprising low cationic pendant organic nitrogen groups and cationic end groups (6B). An example of a viral particulate binding macromolecule composition comprising a cationic core-dimer with PEG-peptide ligand conjugate, 2-high cationic branch macromolecule comprising high cationic pendant organic nitrogen groups plus aliphatic end groups (6C). An example of an antibiotic binding composition comprising a neutral core-dimer, 4-low cationic branch macromolecule comprising low cationic pendant organic nitrogen groups plus cationic end groups (6D).

FIG. 7 depicts a generalized description of modular conjugates and example combinatorial ligand-linker-agent compositions.

FIG. 8 depicts examples of monomers comprising organic oxygen pendant groups.

FIG. 9 shows examples of repeating units and arms comprising organic oxygen pendant groups.

FIG. 10 shows examples of ligand conjugates comprising cyclic RGD peptide and a biologically inactive nucleic acid domain.

FIG. 11 shows an example of ligand conjugates comprising cyclic ROD peptide and doxorubicin agent.

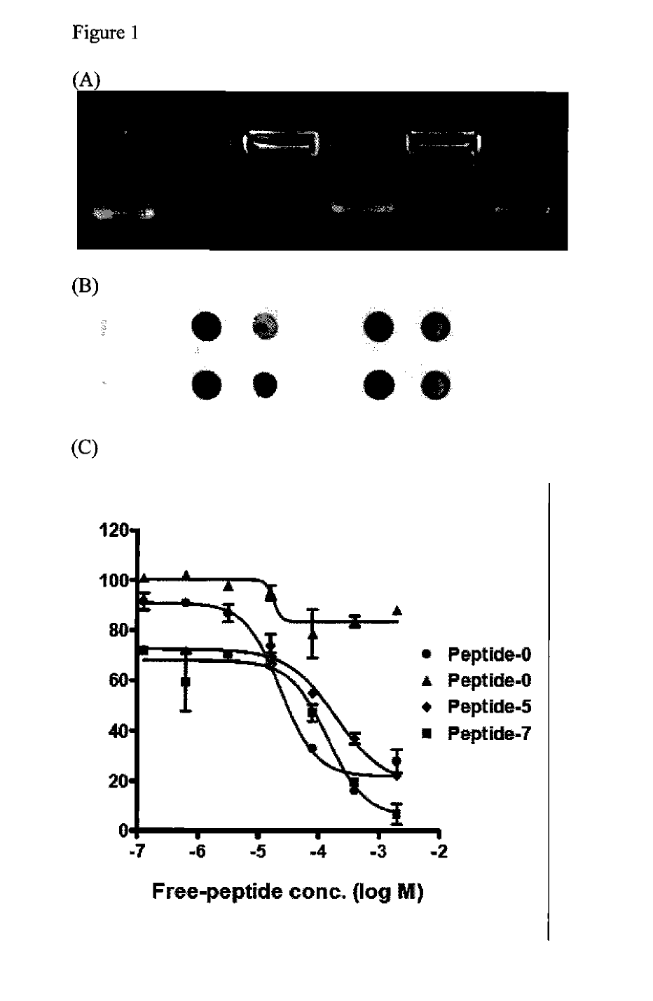

### DETAILED DESCRIPTION OF THE INVENTION

### Definitions

As used herein the following terms are defined as follows:

As used herein “natural amino acid” includes an amino acid found in proteins selected from the group consisting of the L-amino acids: alanine, valine, leucine, isoleucine, praline, phenylalanine tryptophan, methionine, glycine, serine, threonine cysteine tyrosine, asparagine glutamine, aspartic acid, glutamic acid, lysine arginine, proline, and hydroxyproline.

As used herein a “non-natural amino acid” is an amino carboxylic acid that is not a natural amino acid.

As used herein “arm” refers to chemical moiety extending from a core to an optional end group, where the arm permits an interaction with a carrier or carrier-like molecule or a cargo or cargo-like molecule. Where a carrier-like molecule is “like” the carrier in charge or hydrophobicity or hydrogen bonding, or other properties associated with binding and a cargo-like molecule is “like” a cargo in charge or hydrophobicity or hydrogen bonding, or other properties associated with binding (e.g., where a cargo molecule is a polyanion, such as a nucleic acid, the cargo-like molecule is a polyanion such as a nucleic acid that is not the cargo nucleic acid). In some embodiments an arm comprises one or more monomers having optional pendant groups, an optional spacer group(s), optional end group(s) and an optional branch point in the arm. In some embodiments, arms comprising amino acid monomers (e.g., a polyamide) having pendent cationic groups (e.g., amine and imidazole), or anionic groups (e.g., carboxyl or phosphate), or monomers with groups having other activities that will be apparent to the skilled artisan (e.g., thiol, or aromatic groups). In some embodiments of the above described arms the polyamide molecules do not include L-histidine or L-lysine. In other embodiments each arm present in a arm may comprise from about 6 to about 50 amino acids. In still other embodiments, including the embodiments of arms described above, the arms will comprise one to three branches.

In some embodiments, at least about 75%, 80%, 85%, 90%, 95%, 97.5%, 99% or 100% of the pendant groups (e.g., amino acid side chains) present on all of the arms of a carrier are of the same charge (i.e., positive or negative). Optionally the arms can have hydrophobic groups to provide additional sites for hydrophobic and Van der Waals interactions. The arms may also optionally have groups capable of hydrogen binding interactions (e.g., hydroxyl or sulfhydryl, or amide)

As used herein a “core” or “cores” refer to molecules or solids that provide at least two pendant groups upon which arms and other components (e.g., moieties that will form a steric coat) may be attached. Examples of cores include ethylene diamine, 1,2,3-triamino-propane, JEFFAMINE®, amine surface poly amido amide (PAMAM) dendrimer of 1, 2 or 3 generations, branched PEI with a molecular weight less than 5 KD, ethylene diamine tetraacetic acid (EDTA), or a linear or circular polyamide with pendant linkage moieties, or a large number of arms may be attached to a large solid core with many attachment sites on its surface such as thiol binding to the surface of colloidal gold.

As used herein a “nanoparticle-core” refers to the central inner portion of a nanoparticle. In one embodiment the nanoparticle-core may comprise complexes between two oppositely charged materials such as polycationic carrier and nucleic acid or polycationic carrier and polyanionic carrier. In another embodiment the nanoparticle-core may comprise complexes between carrier and poorly water soluble cargo. Where nanoparticles have a solid core the nanoparticle-core may comprise a solid material such as iron oxide or gadolinium containing nanoparticle, colloidal gold, colloidal silver, silica and the like. Solid cores may provide a multitude of attachment sites or pendant groups, such sulfhydryl groups on colloidal gold, e.g., sulfhydryl attachment to colloidal gold, or dextran attachment to iron oxide particles.

Nanoparticles, as used herein have a size of 300 nm or less. In some embodiments, the size may be 100 nm or less. In other embodiments, the size may be 75 nm or less or 50 nm or less.

As used herein the term “branching species” refers to moieties that provide a point where one moiety may branch into two or more, groups or parts (e.g., a polyamide portion of an arm having an amino acid with two or more amine group tails and bound to another amino acid directly or indirectly through the carboxyl group). Branching species includes branching monomers and branching molecules. In some embodiments, branching species include, but are not limited to, neutral compact linkages such as lysine or glutamate or cysteine disulfide, flexible linkage such as with PEG or an aliphatic chain. Branching momomers are monomers found in arms that have several, tails (e.g., two or more, or three or more) for each head, including amino acids such as lysine or diaminobutyrate or carboxy-spermine.

As used herein the term “cargo” describes a compound or other entity that provides all or most of the biological effect or diagnostic signal delivered by the nanoparticles described herein. In some embodiments, cargo includes, but is not limited to compounds or therapeutic agent found in the U.S. Pharmacopeia or Physicians Desk Reference (PDR). In other embodiments, cargo includes, but is not limited to compounds with antiviral, antibacterial (antibiotic), or anticancer (chemotherapeutic) activity, such as doxorubicin, paciltaxol, mitomycin C, 17-AAG, velcade, phosphorylated nucleosides, phosphorylated peptides, and the like. In other embodiments cargo includes experimental or therapeutic or imaging compounds such as: MRI imaging agents containing gadolinium; radio-opaque agents for use with X-ray based analysis (e.g., CAT scan and X-ray images); radioactive compounds and agents used in PET scans (e.g., carbon-11, nitrogen-13, oxygen-18, and fluorine-18); and radio active compounds and agents used in SPECT scans (e.g., iodine-123, technetium-99, xenon-133, thallium-201, and fluorine-18). In still other embodiments a cargo includes but is not limited to a nucleic acid, including but not limited to single or double stranded DNA, RNA molecules. Such nucleic acids include cDNA, mitochondrial DNA, chloroplast DNA, anti-sense DNA, siRNA. and nucleic acid that expresses a detectable protein. In other embodiments cargo includes combinations of entities such as combinations that provide multiple biological effects or diagnostic signals or both.

As used herein, a “carrier” refers to a non-cargo material that packages and stabilizes the cargo, such as a polyamide macromolecule or an oxidized dextran polyacetal with pendant aldehydes, and may subsequently release the cargo at a target cell or tissue. Carriers may provide structure and other key properties to the nanoparticle. In some embodiments carriers comprise arms attached directly or indirectly (e.g., through a linker or spacer) to a core or a solid nanoparticle-core.

As used herein, “ligand” refers to a moiety attached to a component of a nanoparticle that has an affinity for a structure on the surface of a cell or tissue. Embodiments of ligands include, but are not limited to, polypeptides including antibodies, antibody fragments, receptor binding proteins, and small peptides, molecules such as folate, carbohydrates such as sialyl Lewis X.

As used herein “linker” refers to a biodegradable or reversible linkage or group forming a linkage that can be selected from those known to one skilled in the art. Biodegradable linkers include, but are not limited to, amide, ester, carbamate, carbohydrate, and polyacetal. Linkers also include one or more unstable, cleavable or reversible linkages known in the art, including but not limited to, disulfides, esters, polyacetal, vinyl-ether, Schiff base, dithiobenzyl, non-covalent binding peptides, and enzyme recognized peptide sequences.

As used herein, an end group (or end group modifier) refers to a modifier or other moiety appended onto the end of an arm wherein said moiety are known in the art to provide a function not provided by the arm, such as an aliphatic hydrocarbon chain that penetrates into a bilayer lipid membrane or other lipidic material or a membrane fusogenic surfactant.

As used herein, a spacer or flexible spacer refers to any number of flexible agents are known in the art that act as a spacer within the nanoparticles or their component parts. Spacers include, but are not limited to, aliphatic hydrocarbon and ethylene glycol, and a number of hydrophilic agents that act as a spacer and provide a steric layer, including PEG, polyoxazoline, polyacetal, and polysialic acid.

As used herein, “steric coat” refers to a hydrophilic macromolecular surface components on nanoparticles that provide low non-specific binding and/or reduced immunogenicity, such as PEG, oxidized and reduced dextran polyacetal, polyoxazoline, polyglycerol, polysialic acid, polyglutamic acid or hydrophilic polypeptide sequence. Molecules of a steric coat may comprise additionally comprise a linker or ligand.

As used herein, an anchor is a moiety that is carrier like (which means it interacts with cargo) or cargo like (which means it interacts with a carrier or a universal-carrier that is used in the nanoparticle). Because of the interactions, anchors can be incorporated or associated with nanoparticles. In addition to the moiety, anchors comprise a linker that may be used to attach one or more molecules of a steric coat, which may optionally comprise a linker or ligand at a position distal to the anchor moiety.

### I. Modular Polyamide Macromolecules

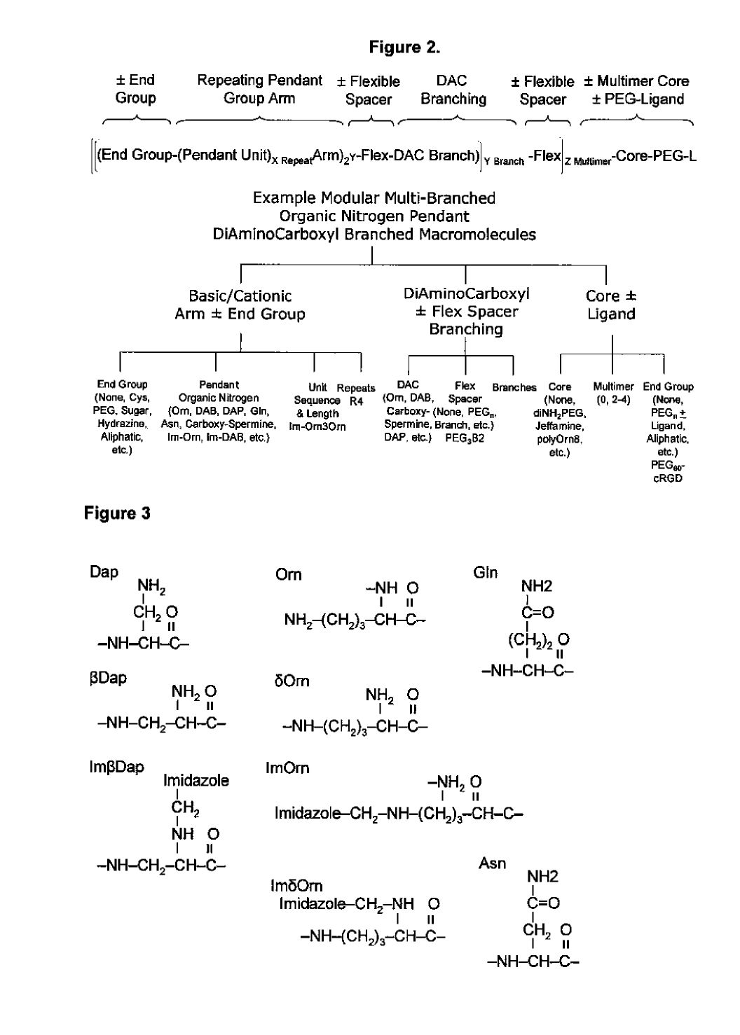

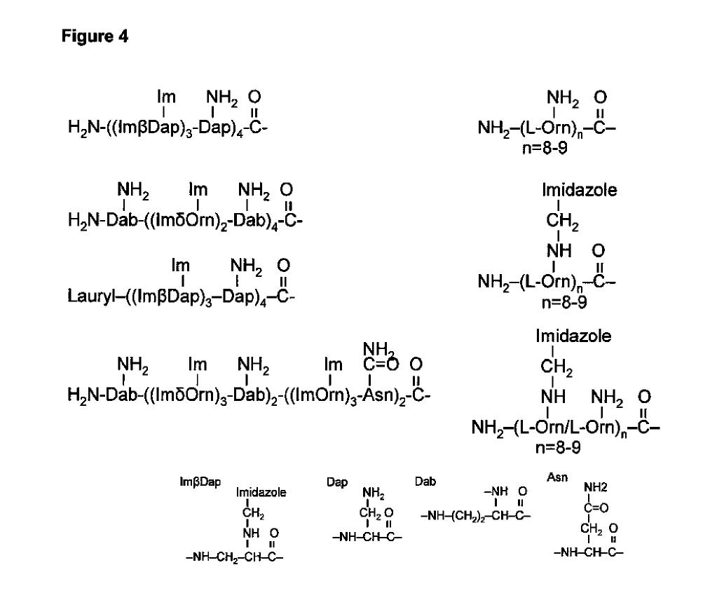

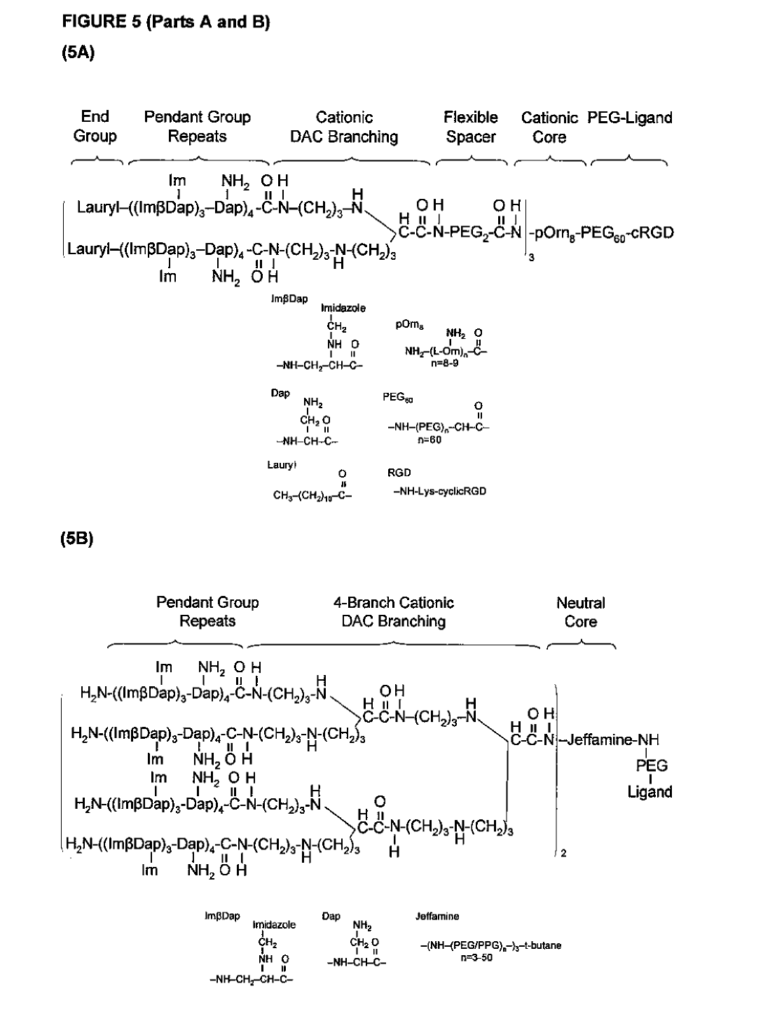

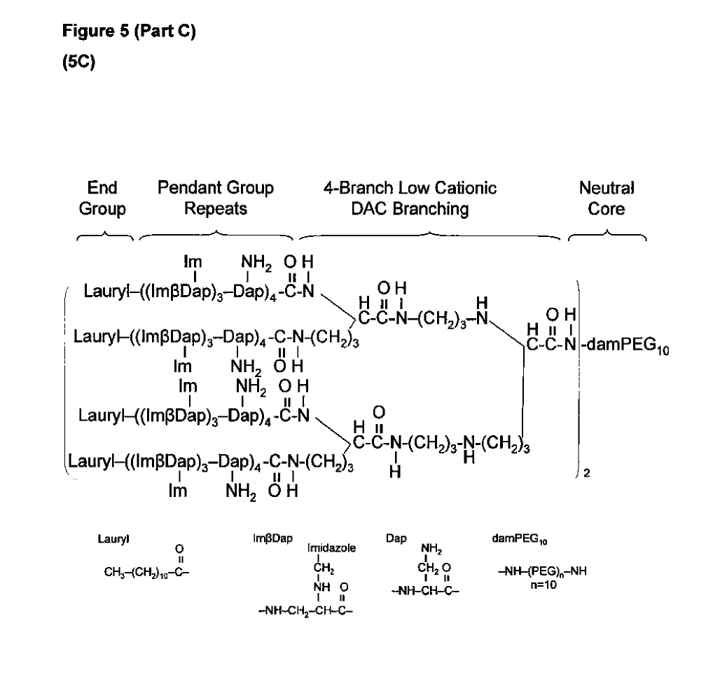

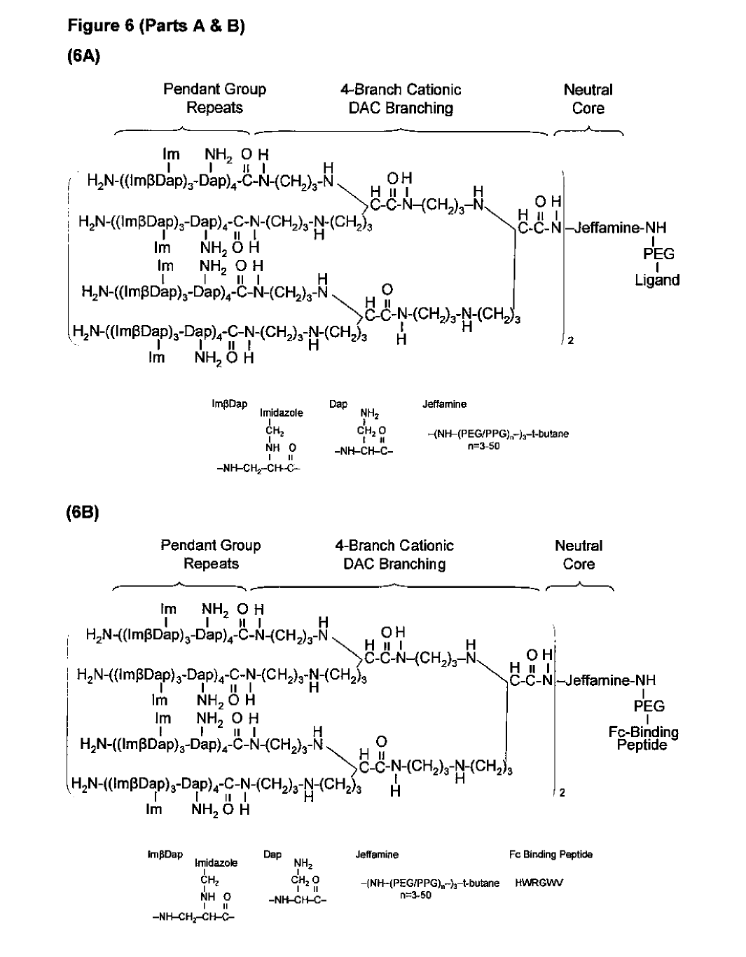

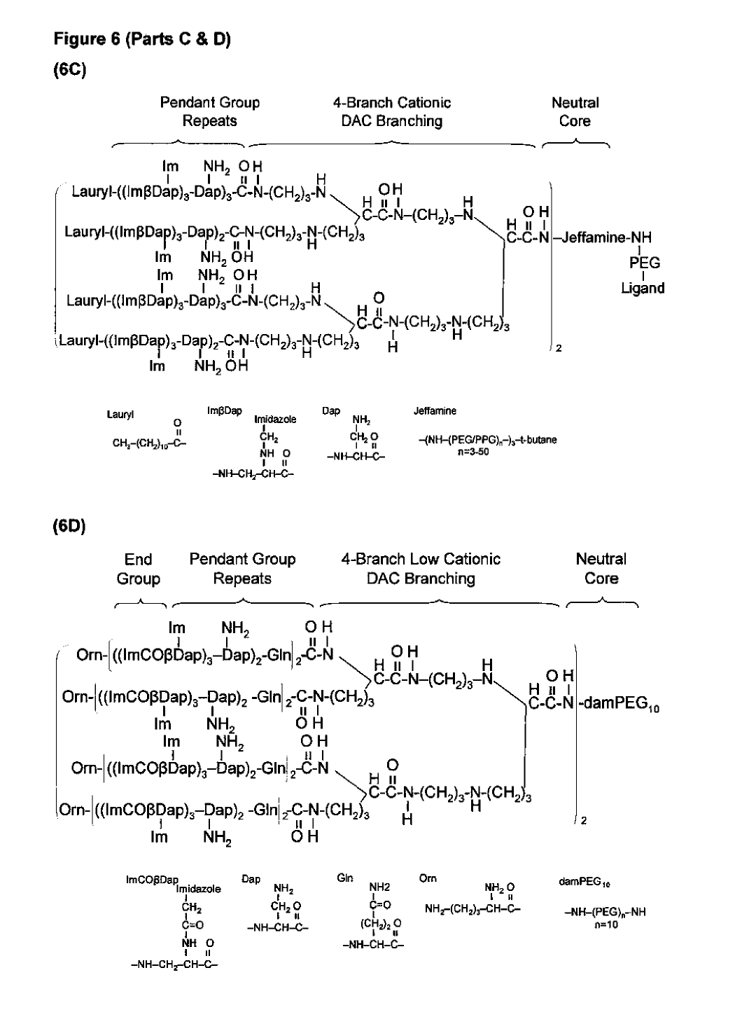

The invention provides modular, optionally branched, polyamide macromolecules comprising organic nitrogen and/or oxygen pendant groups. The compositions provided optionally comprise multimeric macromolecules assembled on a core and optionally further comprise one or more modifying moieties selected from a group that includes a flexible spacer, an end group, a targeting ligand, and a protective polymer. Compositions according to the technology of set forth herein are described in a general formula in FIG. 2 with exemplary combinatorial modular elements for compositions comprising pendant organic nitrogen moieties. The invention provides compositions and methods, for A) monomers, and other modular components, B) biodegradable linkers and/or optionally reversible linkers, C) arms and their assembly, D) optional assembly of branched macromolecules, E) optional core and linker compositions for multimers, F) optional modifiers, G) production for commercialization and H) biomedical applications. The methods of preparing and testing the modular molecules may be conducted in a combinatorial fashion. FIG. 5 shows macromolecular compositions illustrating the molecular diversity of the macromolecules of the invention. FIG. 6 provides specific compositions for specific biomedical applications illustrating matching macromolecules of the invention with biomedical applications. Section IV discloses several compositions and methods for specific biomedical application of those compositions. One of ordinary skill in the art will recognize that the invention is not limited to these reagents, monomers, repeating units, arms, macromolecules, multimers, ligands and other modifiers, and biomedical applications or examples disclosed.

In one embodiment, macromolecular compositions are provided that complex nucleic acid agents or analogues. While polymeric nucleic acid carriers have been described such as PEI, dendrimers, and recently histidine-lysine copolymers, it is now appreciated that nucleic acid delivery requires higher cationic charge density and in vivo application requires low toxicity and biodegradability, yet none of the previous polymer systems fulfill all these requirements. Also, none of the previous carrier systems permit adjustment of the chemical structure so that tuning to cargo or combinations of cargo is possible.

One embodiment, described in FIG. 6A, provides a cationic branched macromolecule with higher charge density yet biodegradable due to a polyamide backbone, and further comprising an integrin targeting ligand peptide coupled through a protective PEG. This composition is synthesized by a series of steps. To prepare monomers comprising imidazole pendant moieties, imidazole carboaldehyde may be reacted with delta-Boc diaminobutyrate and the resulting Schiff base reduced with cyanoborohydride to form the secondary amine linked imidazole of DAB. Then the imidazole is protected, and finally the carboxyl activated to the NHS ester. This product and alpha-Boc delta amino protected, e.g., Fmoc, DAB with carboxyl activated NHS ester are used in solid phase synthesis to produce tetrapeptides and cleaved from the resin without removing the protecting groups. The carboxyl of this repeat unit is activated to give the NHS ester and then they are coupled to give pairs of repeat units and then to give arms comprised of four repeat units, and again with all pendant groups remaining protected. The coupling can be performed with Boc protection of coupling amines and orthogonal, e.g., Fmoc, protection of the pendant groups. The carboxyl of the arms is activated to give the NHS ester and then two arms coupled to each of two deprotected amino groups of each ornithine, and again with all pendant groups remaining protected. The carboxyl of the branched composition is activated to give the NHS ester and two reacted with each ornithine previously coupled at its carboxyl to PEG60 coupled at its distal end to cRGD, and finally the four branched macromolecule comprising PEG-ROD ligand subjected to protection group removal followed by precipitation and diafiltration purification.

Other compositions forming complexes with nucleic acids or analogues comprise one or more of 1) at least one carboxyspermine branching species and more preferably at least three, 2) at least dimers of 4 branched macromolecules and more preferably at least trimers, 3) at least one neutral or zwitterionic hydrophilic polymer and in preferred embodiments at least one for 4 arms, and in one preferred embodiment said polymer being selected from the group of PEG, oxidized and reduced dextran polyacetal, polyoxazoline, and in another preferred embodiment further comprising an reversible or cleavable linkage, 4) peptide ligand coupled through a PEG linker of at least 3 KD molecular weight, and in one preferable embodiment the peptide comprises a cyclic ROD peptide with selective binding affinity for integrins associated with neovasculature, and in another preferred embodiment further comprising an reversible or cleavable linkage, and 5) at least one additional ligand with different binding affinity.

A. Monomers, Arm End Groups And Other Modular Components

The invention provides monomers with heterobifunctional moieties and their assembly in a head-to-tail fashion to form macromolecules. Monomers are also provided that comprise two or several tails for each head that form branches where they are incorporated. Heterobifunctional monomers are also provided with a carboxyl group as the head and one amine at the tail, where the amine and any reactive side group is protected and the carboxyl is activated to react with an unprotected amine of another monomer or multimer. Monomers, repeating units comprising monomers, and arms comprising one or more monomers that may used in the preparation of nanoparticles described herein include, but are not limited to:
solid phase peptide synthesis amino acid reagents (including:
   Boc-Fmoc-Ornithine; di-Boc-Ornithine, Boc-Fmoc-Dab, di-Boc-Dab, Boc-Fmoc-Lys; di-Boc-Lys, α-amino-Boc amino acids carboxyl NHS activating reagent, and amine protecting reagent)
imidazole-4-acetic acid monohydrochloride
di-Boc-guanidium-acetic acid
imidazole-carboaldehyde
Boc-N-(Boc-N)Lys-(Boc-Imidazole)His-(Boc-Imidazole)His-Carboxyl
H2N-(Boc-N)Lys-(Boc-Imidazole)His-(Boc-Imidazole)His-Carboxyl

Reagents and monomer compositions comprising protected amine and activated carboxyl groups useful for the preparation of the nanoparticles described herein also include, but are not limited to:
Boc-Imidazole-4-acetic-NHS
Boc-Imidazole-methylamide-orinithine (α- or δ-); Boc-Imidazole-methylamine-orinithine (α- or δ-)
di- or Tetra-Boc-5-carboxy-spermine

Reagents and monomer compositions comprising a flexible spacer useful for preparing the nanoparticles described herein also include, but are not limited to:
ω-amino-Fmoc/Boc-PEGn-carboxylic acid, n=2-27
Boc-ω-amino-Fmoc/Boc-PEGn-carboxyl-NHS ester, n=2-27

The invention also provides reagents and compositions comprising end groups:
fatty acid-dianhydride and aliphatic-amine
all-amino/imidazole Boc segments: His3O—OH, ImO3O—OH, ImδO3O—OH and ImδO3δO—OH
ligand-carboxyl, ligand-amine, Ligand-PEG-carboxyl, Ligand-PEG-amine (e.g., peptide ligands such as cRGD-Lys-NH₂)
linker-PEG-carboxyl, linker-PEG-amine (e.g., linker such as hydrazide or mAb Fc binding peptide)

B. Biodegradable Linkage And Biochemically Reversible Linkage

The technology describe herein provides for linkages between modular elements, including linkages to end groups, targeting ligands, covalent or non-covalent linkers for oriented antibodies such as hydrazide linkage to oxidized antibody glycosylation or an Fc region binding peptide, and protective hydrophilic polymers and similar groups. The linkages may be a biodegradable linkage that may be selected from those known to one skilled in the art. Such linkages including stable linkages such as amide, ester, carbonate, carbamate, carbohydrate, and polyacetal, or unstable, cleavable or reversible linkages, including, but not limited todisulfide, ester, polyacetal, vinyl-ether, carbamate, Schiff base, dithiobenzyl, and enzyme recognized peptide sequences.

C. Arms Comprising Pendant Groups

The present technology provides arms comprising one or more monomers with organic nitrogen or oxygen pendant groups or their combination. The invention optionally provides arms comprising one or more monomers with other activity pendent groups such as thiol, or hydrophobic or aromatic moieties. Repeating units and arms optionally may further comprise a spacer, an end group, and/or branching within the arm. In some embodiments, arms comprise a linear sequence, or non-linear structure and further optionally comprising unique chemical properties (groups) at one or both ends. In other embodiments the arms have a defined structure, or alternatively a polydisperse structure. In one embodiment, the arms comprise a repeating sequence of pendant imidazole and primary amine groups and optionally may further comprise hydrophilic amide, hydroxyl, or carboxyl pendant groups or their combination. Other embodiments provide for arms that further comprise one or more flexible spacers and optionally a cleavable or reversible linkage. The invention provides arms comprising monomers comprising organic nitrogen pendant groups, including:
N-terminal Boc Asn with carboxyl NHS ester activation (solid phase synthesis or combined solid phase/solution phase synthesis and solution phase. NHS activation)
N-terminal Boc, amine and imidazole protected (ImO3O)4-OH with carboxyl NHS ester activation,
N-terminal Boc, amine and imidazole protected (ImδO3O)4-OH with carboxyl NHS ester activation and
N-terminal Boc, amine and imidazole protected (ImδO3 δO)4-OH with carboxyl NHS ester activation
N-terminal Boc, amine and imidazole protected (His3O)4-OH with carboxyl NHS ester activation (solid phase synthesis or combined solid phase/solution phase synthesis and solution phase NHS activation)

D. Branched Macromolecules With Optional Flex Spacers

The invention provides branched macromolecules that may optionally further comprise within branches one or more flexible spacers including PEG or polyacetal regions. In one embodiment, the invention provides branched macromolecules comprising sequences of monomers coupled by biodegradable linkages and optionally further comprising one or more cleavable or reversible linkages. The branched macromolecules optionally may comprise a spacer. In one embodiment amide linkages are provided by sequential addition of diaminocarboxylate compounds (DAC) with protected amino groups and activated carboxyl group followed by deprotection of the amino groups for addition of other chemical substituents. In preferred embodiments, the invention provides branched macromolecules with specific chemical properties at their ends.

E. Optional Multimer Core With Optional Modifiers

The invention optionally provides multimers with multiple macromolecules coupled together (e.g., core to core), where the multimer optionally comprises one or more modifiers such as an end group, targeting ligand, flexible spacers, and reversible or cleavable linkages (e.g., PEG, oxidized and reduced dextran polyacetal, polyoxazoline, polyglycerol, polysialic acid, polyglutamic acid or hydrophilic polypeptide sequence). In one embodiment amide linkages present in the arms of a carrier or a multimer are provided by sequential addition of activated macromolecule carboxylate compounds comprising protected amino groups and activated carboxyl group to a core comprising two or more amine groups followed by deprotection of the macromolecule amino groups. In some preferred embodiments, the invention provides cores comprising unique chemical properties. The invention also provides cores comprising a solid material such as iron oxide nanoparticles or colloidal gold that provide a multitude of attachment such as dextran attachment to iron oxide or sulfhydryl attachment to colloidal gold. In addition, the invention provides cores that also contribute a desired activity such as iron oxide nanoparticle imaging agents.

The invention optionally provides multimer branched macromolecules comprising commercially available cores with two or more attachment sites, including:
Huntsman Jeffamine—(amino-PPGn)₃ and amino-PPG-PEG-PPGn-amino: T-403, T-3000, D230, D-400, ED-600, . . .
diaminoPEG (H2N-PEGn-NH2): n=7-100
Poly-ornithine (pOn): 1 KD
cyclic polyornithine
amine surface PAMAM dendrimer of 1, 2 or 3 generations
Polyethyleneimine: readily available in branched form with average molecular weight from 0.5 to 25 KD.
colloidal gold nanoparticles
iron oxide nanoparticles

F. Optional Modifier

Macromolecule compositions are provided that optionally comprise one or more modifiers. In one embodiment, compositions further comprise end group modifiers, where said end groups can be one or more species selected from the group of ionizable, hydrophobic, and hydrophilic groups, and ligands. End groups may be bound through non-covalent linkage or through covalent biodegradable linkages that are stable, reversible, or cleavable. Ligands can be selected from those known to one skilled in the art such as polypeptides including antibodies, antibody fragments, receptor binding proteins, and small peptides, molecules such as folate, carbohydrates such as sialyl Lewis X. The invention provides peptide ligands providing selectivity for targets exposed on the surface of cells and in one preferred embodiment provides ligands for cell surface targets accessible to compositions of the invention administered directly to the blood, such as RGD peptide ligands targeting neovascualture, galactose or malarial surface protein peptides targeting hepatocytes, and ligands targeting circulating lymphocytes. Modifiers may be bound through a non-covalent linkage, such as a biotinylated moiety binding to an avidin or similar protein, an antibody ligand bound to a modifier comprising a peptide which binds the Fc fragment of an antibody, such as HWRGWV, HYFKFD, HFRRHL, and HVHYYW, disclosed in U.S. Pat. No. 7,408,030, which is hereby incorporated by reference. Alternatively, modifiers may be bound through covalent biodegradable linkages that are stable, reversible, or cleavable. In a preferred embodiment, ligands are bound in a non-random orientation, such as by covalent coupling to antibodies at a glycosylation site. In another embodiment, macromolecule compositions further comprise one or more hydrophilic polymer such as PEG, oxidized and reduced dextran polyacetal, polyoxazoline, polyglycerol, polysialic acid, polyglutamic acid or hydrophilic polypeptide sequence. Said hydrophilic polymer may be bound through non-covalent linkage or through covalent biodegradable linkages that are stable, reversible, or cleavable. In another embodiment, macromolecule compositions further comprise one or more hydrophobic modifiers such as an aliphatic hydrocarbon useful to interact with cell membranes or a viral envelope. Other optional modifiers are provided by the invention, understood by one with skill in the art.

G. Synthesis

The present disclosure provides for improved and less costly synthesis and manufacture of the nanoparticles described herein. In one embodiment, the macromolecule is synthesized stepwise. In another embodiment, monomers are assembled into repeating units and then repeating units are assembled into macromolecules, and optionally macromolecules become arms assembled into branched macromolecules with either homogeneous or heterogeneous arms, and optionally branched macromolecules are assembled into multimers. In another embodiment, production is provided by combinatorial synthesis. In still another embodiment, synthesis of defined macromolecules comprising monomers comprising organic nitrogen pendant groups is provided for. Also provide for is the production of monomers, end groups, and other elements, the production of units, arms, cores, and other elements by combinations of commercially available raw materials and produced raw materials, and the production of macromolecules by combinations of arms, cores, end groups, and other elements.

In one embodiment amide linkages are provided by sequential addition of aminocarboxylate monomers with protected amino groups and activated carboxyl groups followed by deprotection of the amino groups for subsequent reaction with another monomer, optionally using solid phase synthesis. In some embodiments assembly of monomers by said sequential amide linkage formation using solid phase synthesis produces repeating units. In other embodiments, assembly of arms from amide linkage of repeating units is performed using solution phase synthesis, for optional assembly of branched macromolecules or multimers.

In another embodiment the monomers couple to an initiator or to the end of a growing polymer such as by a ring opening linkage, where degree of polymerization and the molecule weight are controlled by the initiator to monomer ratio. In another embodiment monomers or repeat units are assembled into macromolecules by a head to tail coupling with a ratio of species reactive at both head and tail with species reactive at only head or tail where the latter species terminates the polymerization at either the head or tail. The controlled ratio of all three species provides a polydisperse macromolecule with a low heterogeneity.

(i) Monomers with Pendant Moieties and Protected Forms

In one embodiment, the invention provides for monomers comprising hetero-bifunctional compounds having different reactive groups on the two ends of the monomer, such as aminocarboxylates, and further comprising organic nitrogen pendant groups, optionally where the pendant groups are protected. For embodiments when monomers comprise aminocarboxylates, diverse peptide synthesis reagents are available for use in the invention, including species with pendant groups comprising moieties useful for the invention such as amine, imidazole, amide, hydroxyl, carboxyl, thiol, and aliphatic or aromatic species and can be provided by natural or non-natural aminocarboxylates, (i.e. amino acids.). One embodiment, provides for non-natural monomers with pendant group comprising imidazole such as 2-methyl histidine or imidazole coupled to a diaminocarboxylate via an amide linkage, such as coupling the NHS ester of 4-acetic acid-imidazole to a monomer with unprotected primary amine, a secondary amine linkage, such as coupling 4-formyl-imidazole or 2-formyl-imidazole to a monomer with unprotected primary amine followed by mild reduction of the Schiff base to the secondary amine, or a carbamate linkage, such as coupling of 4-acetic acid-imidazole to a monomer with unprotected primary amine with carbodiimidazole.

(ii) Arms

The present disclosure provides for biodegradable macromolecular arms. In one embodiment the disclosure provides compositions and methods for synthesis of repeating units or arms by forming couplings of monomers, or in other embodiments assembly of repeating units into arms. The arms can be formed exclusively from monomers comprising organic nitrogen pendant groups, or from monomers comprising other pendant groups or no pendant group. Arms can be synthesized by coupling monomers or repeating units comprising hetero-bifunctional moieties, such as ones with an amino group at one end and a carboxyl group at the other end. Arms may further comprise one or more PEG spacer and/or end group modifiers. In one embodiment, arms are formed by amide linkage of DAC monomers.

In one embodiment, repeating units can be assembled into arms by a coupling that connecting the head of one unit to the tail of another by solid phase synthesis or by solution phase synthesis. When a single assembly is desired, or when the method encounters steric hindrance limitations production is preferably by solution phase synthesis. In a related embodiment, repeating units are palindromic with respect to the amino acid sequence but still have one N-terminal end and one C-terminal end providing head to tail assembly. In another embodiment, repeating units are assembled so that the arm lacks a palidromic sequence or is polydisperse lacking a defined structure.

(iii) Biodegradable Branched Macromolecules

The present disclosure includes and provides for biodegradable branched macromolecules. Also provided for are compositions and methods for the assembly of arms on to a core forming a branched structure, in one embodiment by coupling one end of each arm to a core. The branching species within a core can be a linear segment of monomers such as where pendant primary amino groups are available for coupling. Alternatively, they can be non-linear (e.g., such as where a species comprises two or more terminal primary amino groups available for coupling, dendrimers or branched PEI).

In other embodiments one or more arms are coupled to the core with covalent but biodegradable or reversible linkages, such as amide, ester, carbamate, polyacetal, hydrazone, vinyl ether, disulfide, dithiobenzy. One embodiment provides for the synthesis of branched macromolecules through coupling arms to a branching species by forming ester or carbamate bonds between carboxyl moieties and alcohol moieties on a branching species, such as to a linear peptide comprising serine residues. In yet another embodiment, the carboxyl head of arms comprise hydrazine moieties and are coupled to a branching species comprising aldehyde moieties such as an oxidized polysaccharide. In yet another embodiment, the branching species comprises pendant moieties such as carboxyls or amines that further comprise terminal maleimide moieties coupled to arms comprising at least one sulthydryl moiety. In one such embodiment the arm comprise a single sulthydryl moiety. Other linkages are provided, such as reversible dithiobenzyl linkage, known to one skilled in the art.

In one embodiment branching species comprise amino-carboxyls, and synthesis is carried out using peptide synthesis reagents, including species to form branches that have two tail groups for each head group. The invention also provides for production of branches with internal groups or pendant groups comprising groups useful for the invention such as secondary amine, alcohol, PEG, and enzymatically cleavable peptide sequences. In one embodiment cationic branched macromolecules are formed by amide linkage of the carboxyl of carboxy-spermine or carboxy-spermidine to a primary amine of another carboxy-spermine or carboxy-spermidine. Such cationic branched macromolecules may further comprises one or more PEG spacers. In another embodiment branched carboxyl-spermine macromolecules are coupled by a carbamate linkage between the carboxyl and the secondary amine, and optionally further comprises one or more PEG spacer. In yet another embodiment cationic branched macromolecules are formed by amide linkage between ornithines.

The technology described herein also provides for production of branched macromolecules with arms coupled to a core. The arms may be attached by sequential addition of monomers, or by addition of fully assembled arms or large sections of the arms. In one specific embodiment, a carboxyl of an arm is activated as NHS esters and mixed with cores with primary amines, where any other pendant organic nitrogen (e.g., amine, amide, imidazole or hydrazide) present on the core is in a protected. Once the arms have coupled to the core, if a multimer is not to be prepared then all protective groups can be removed.

(iv) Multimers

The present disclosure provides for the production of optional multimers, comprising macromolecules coupled to a core. In one embodiment, multiple branched macromolecules, containing protected pendant groups, are coupled to a core followed by deprotection of the pendant groups and purification of multimers. The invention also provides macromolecules optionally linked to a core to form multimers through stable biodegradable linkages such as an amide, ester or similar linkage, and optionally comprising one or more reversible or cleavable linkages such as hydrazone, vinyl ether, disulfide, or dithiobenzyl. The core may further comprise one or more modifier groups such as PEG, PEG-ligand, or an aliphatic moiety.

(v) Combinatorial Compositions

The invention optionally provides for synthesis of combinatorial compositions. In one embodiment, modular moieties are assembled using combinatorial synthesis techniques known in the art to produce macromolecular compositions with pendant organic nitrogen, and optionally libraries of such compounds. An exemplary combinatorial macromolecule composition is shown in FIG. 2.

(vi) Optional Modifiers

The present disclosure provides for the production of macromolecule compositions that comprise one or more modifiers. In one embodiment, modifiers are incorporated at the last step in the synthesis. In other embodiments, modifiers are incorporated into arms, branched macromolecules, or core moieties, followed by further synthetic assembly. The present disclosure also provides for the production of activated modifiers and reagents to couple modifiers to modular moieties or macromolecules described herein. The disclosure further provides for the coupling of modifiers through stable biodegradable linkages that optionally comprising one or more reversible or cleavable linkages.

### II. Surface Decorated Nanoparticles

The invention provides conjugates of a hydrophilic material for surface decoration of a nanoparticle comprising a cargo, where the surface decoration provides one or more functions such as stability, protection from enzymes and other agents, reduced immunogenicity, avoidance of blood clearance, and sites for attachment of ligands and other surface functionalities. The invention optionally provides for nanoparticles with one or more cargos, including micelles, microemulsions, liposomes, and polymeric colloids. The nanoparticles optionally comprise polyamide macromolecules. The nanoparticles also optionally comprising surface decorations such as hydrophilic polymers providing steric protection and exposed ligands providing cell and tissue specific targeting. Surface moieties are effectively attached in a manner that 1) overcomes past problems where their attachment to carrier distorts its required interactions with cargo for loading or interactions required for nanoparticle formation, and 2) so that surface moieties are shed when cargo is released by disassembly of the nanoparticle, rather than shedding of surface moieties. In prior nanoparticles comprising surface moieties, a standard composition has utilized attachment of the surface moieties through conjugation to carrier material that binds or entraps cargo, such as the lipids of liposomes or cationic polymers forming nanoparticle complexes with nucleic acids. When a bulky hydrophilic material such as PEG or a targeting protein such as transferrin is used for surface modification, this attachment can help limit particle size growth. However, this may also diminish carrier interactions with cargo, which may have substantial deleterious effects, such as decreasing loading efficiency, decreasing particle stability, and accelerating premature cargo release. To solve this problem, the technology described herein provides for orientation of surface moieties and in some embodiments oriented conjugation instead of random conjugation to one or more of many possible sites.

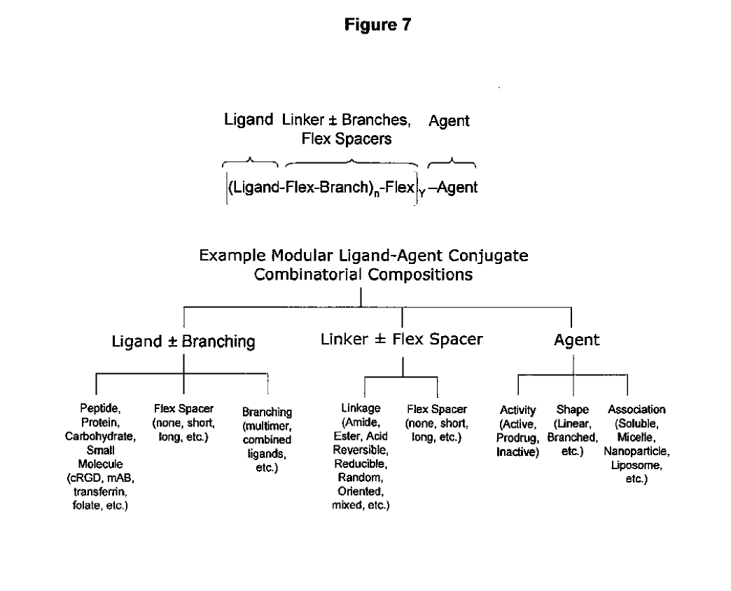

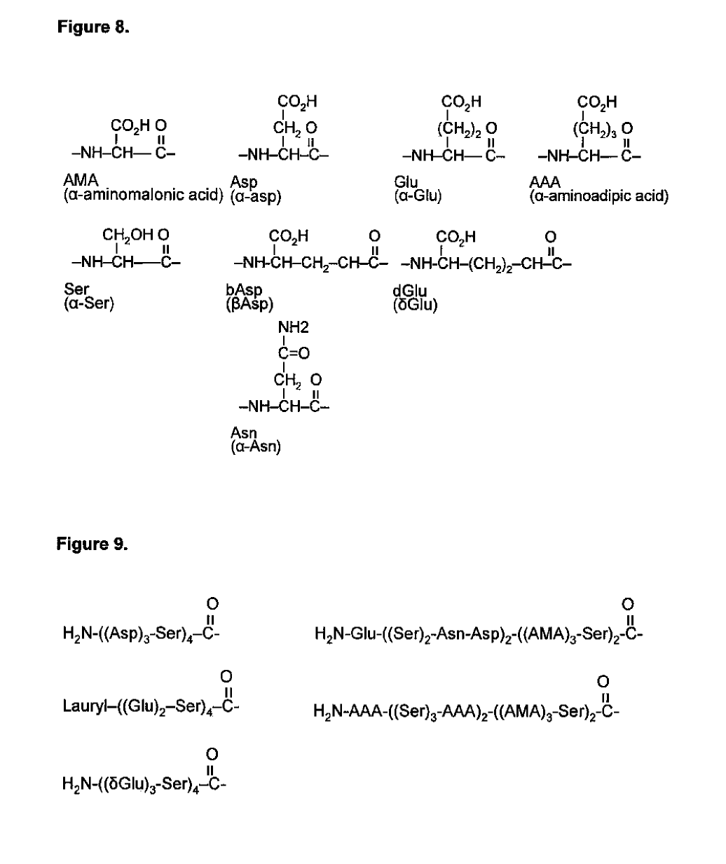

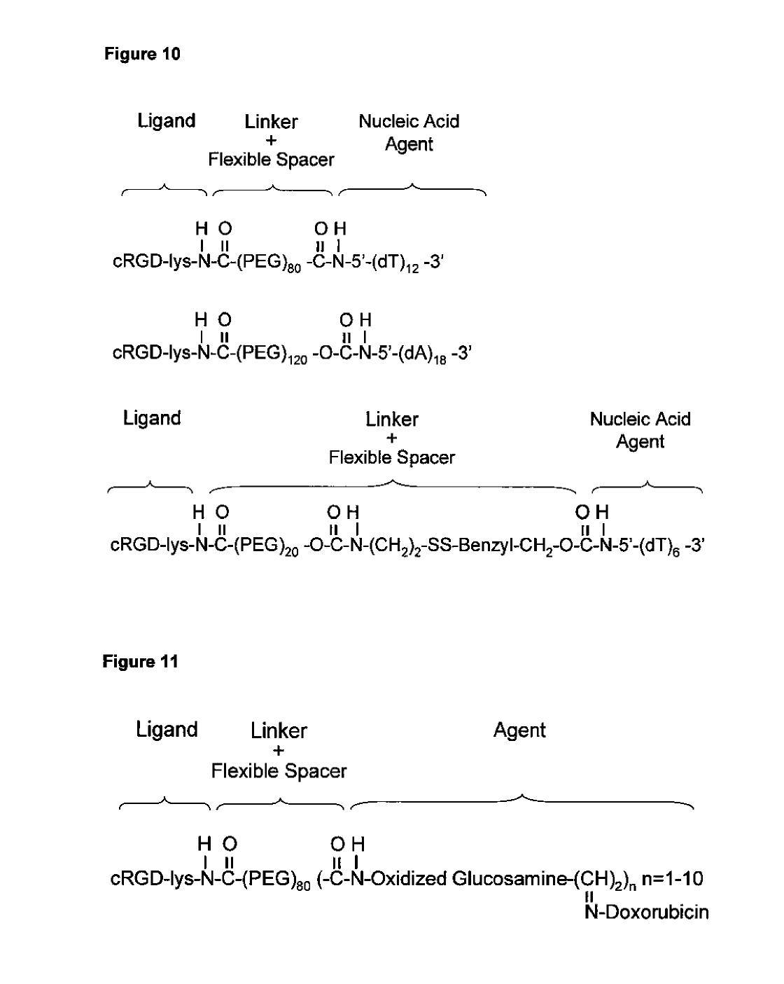

The technology described herein provides modular, optionally branched, conjugates comprising ligand or linker and either carrier or a cargo or both. The compositions provide macromolecule or nanoparticle compositions, and optionally multimeric macromolecules and optionally further comprise one or more modifying moieties selected from a group that includes flexible spacer, modifier functional groups, protective polymer, and optionally reversible linkage. Compositions according to this embodiment are shown in a general formula in FIG. 7. The invention provides compositions and methods of preparing a) ligands, b) linkers and optionally reversible linkers, c) optional flexible spacers and modifiers, and d) anchors, which may be combined and tested in a combinatorial fashion.

The technology described herein provides ligands and other modular components, that in some embodiments can be used to direct nanoparticles to a cell displaying a specific receptor or binding component for the ligand. In one embodiment, the ligands comprise a binding functionality and at least one moiety for conjugation such as amine, carboxyl, hydroxyl, or aldehyde, where other moieties for conjugation preferably are protected so their reactivity blocked. Ligands useful for the invention are known to one skilled in the art, such as peptides such as cRGD-Lys-NH2, phage display peptides, small molecules such as galactose, sialyl Lewis X, or vitamins such as folate, proteins such as antibodies and their fragments, agonist and antagonists of cell receptors and peptide fragments such as segments of malaria surface factor, transferrin, Tenascin C, VEGF, Epithelial Growth Factor, carbohydrates such as sialyl Lewis X, and modified citrus pectin (MCP). The invention provides compositions comprising at least one non-covalent linkage, between an antibody and a antibody binding moiety, such as provided by an antibody binding peptide, and optionally further comprising coupling of said moiety to molecule of a steric coat (e.g., a PEG) that is coupled at its distal end to an anchor, wherein said antibody binding moiety can non-covalently couple an individual antibody, a fragment thereof, or a cocktail thereof (e.g., two or more different antibodies).

A. Oriented Attachment To Carrier-Like Material

In one embodiment, the technology described herein results in an oriented conjugation of the surface moiety to a fragment of a carrier material or preferably another material that is substantially similar to carrier, both referred to here as carrier-like. In some embodiments the conjugation occurs through a single site of attachment of surface moiety to the carrier-like material, where said carrier material is linear, i) said attachment site is preferably at or near one end, ii) the carrier-like material may represent from about 5% to 50% of the size of a carrier molecule, and iii) optionally anchor has a higher density of interactions with cargo than carrier. The same considerations apply for cargo-like anchors. In this embodiment, the surface moiety may be conjugated to one end of a linear carrier-like polymer, such as to the carboxy-terminal end of a homopolymer of cationic amino acids, or to the amino-terminal end of an anionic side chain homopolyamide. A surface moiety may be conjugated to a moiety incorporated at specific positions within a carrier-like material, such as to thiol or other pendant moiety selectable by an orthogonal chemical conjugation reaction, inserted into a cationic side chain homopolyamide, in one instance by linking together small sections of cationic homopolyamide with a linker providing said pendant group or in another instance by sequences defined during solid phase synthesis. A surface moiety may be conjugated to a lipid moiety compatible with lipids used to form a nanoparticle, such as DSPE or POPE, and incorporated into a lipid layer on the surface of a nanoparticle by mixing with other lipids prior to formation of nanoparticles or mixing with preformed nanoparticles, optionally at a temperature near or above a (gel to liquid crystal) phase transition and then cooling.

In one embodiment, the invention provides for cyclic ROD peptide comprising a lysine conjugated to the carboxyl group of an H₂N-PEG-CO₂H with its distal amine conjugated to the carboxyl of a polyornithine of about 2000 MW. Such compositions are useful for the preparation of neovasculature targeted siRNA nanoparticles.

In another embodiment, the invention provides for folic acid conjugated to one end of PEG with its distal end conjugated to the end group thiol formed by thiolysis of a cationic polyacetal, such as that formed by oxidation of dextran followed by coupling of pendant amines via the multitude of aldehyde moieties, Such compositions are useful for the preparation of tumor targeted gene therapy nanoplex comprising a gene expression cassette and a histidine-lysine copolymer carrier.

In yet another embodiment, the invention provides for a hydrazide moiety conjugated to one end of PEG with its distal end conjugated to the carboxyl terminal end of PLGA. Such compositions are useful for the preparation of targeted nanoemulsion or albumin nanoparticle preparations of poorly water soluble chemotherapeutics. The nanoemulsion or nanoparticle may be decorated by coupling of oxidized antibody to surface exposed hydrazide moieties.

In another embodiment, the conjugates comprise “anchors” that bind unconjugated cargo within a nanoparticle, by non-covalent bonds, and thereby modify the biochemical properties of the unconjugated agent to reduce solubility, make the particle electrostatically approximately neutral, reverse the particle's electrostatic charge, reduce its toxicity or otherwise modify the cargo to achieve a desired property. Examples include conjugates comprising ionic peptides, single stranded nucleic acids, or their analogues, that bind unconjugated neutral nucleic acid analogue cargo where the binding conveys electrostatic charge onto the unconjugated nucleic acid cargo. Use of this embodiment includes loading one or more unconjugated uncharged cargo in targeted nanoparticles comprising an oppositely charged nanoparticle forming material carrier. Examples of charged agents include nucleic acid or analogues comprising RNA, DNA, nucleic acids with modified backbone and/or bases, poly amino-carboxyl-monomers with pendant carboxyl or other organic oxygen moieties, polysialic acid and analogues, cationic antibiotics, ionizable drugs.

In one embodiment, conjugates are provided that comprise antibody ligands coupled by reversible or non-covalent bonds to a linker, which is bound to a molecule of a steric coat. One embodiment is directed to a composition that comprises an antibody coupled by non-covalent binding to a linker, such as an antibody bind peptide, where the antibody binding peptide or linker can non-covalently couple any individual antibody or mixture of antibodies or fragment or analogue comprising the antibody binding region. Efforts to develop methods to purify monoclonal antibody using chromatographic techniques have identified a number of short peptide sequences that bind to the Fc region of antibody molecules and which can be used as antibody binding peptides. In the present embodiment, such peptides can be used to couple antibody molecules to polymers and nanoparticles through non-covalent interaction.

Peptides binding to the Fc region of the antibody will not affect the antigen binding ability of the antibody and will be able to bind a variety of antibody molecules. Macromolecules, multimers and nanoparticles with antibody binding peptides exposed on the surface provide a platform to coupling any antibody of interest. This platform allows targeting of macromolecules polymers and nanoparticles to various tissues and cells, if an appropriate antibody is available. This provides a method to target therapeutic agents to disease tissue and cells thereby minimizing toxic side effects. An example of a composition, shown in FIG. 6B, comprises at least one antibody binding moiety, such as an antibody binding peptide to a PEG or other flexible linker that is coupled at its distal end to a macromolecule or multimer of the invention, where said antibody binding moiety can covalently or non-covalently couple an individual antibody or fragment or a cocktail thereof. Antibody binding peptides include those identified to bind the Fc region, such as the sequences R-PEG-CO—HN-HWRGWV-CONH₂, HCO—HN-YYWLHH-CONH-PEG-R, other sequences described in U.S. Pat. No. 7,408,030, or other peptide sequences such as HWRGWVC, HWRGWA, HWRGWA, HWRGWL, HWRAWV, HFRRHL, HFRRHI, HFRRHA, HVHYYW, HAHYYW, and HYFKFD.

Other embodiments provide compositions comprising at least one covalent antibody linkage such as i) a biodegradable conjugation linker for reactive moieties exposed on an antibody or fragment or ii) biodegradable conjugation linker for a biochemical modification of an antibody or fragment such as an oxidized antibody glycosylation. In other embodiments the technology provides non-covalent linkage of an antibody by protein A or of biotinylated antibody by avidin. Other examples of compositions comprising antibody ligand comprise one or more of, at least one antibody or fragment or analogue bound.

B. Oriented Attachment To Cargo-Like Material

In another embodiment, the invention provides oriented conjugation of a molecule of the steric coat to a cargo-like material or another material that is substantially similar to cargo, both referred to here as cargo-like, preferably with a single site of attachment of surface moiety to the cargo-like material; optionally wherein i) the cargo-like material is linear, ii) the attachment site is at or near one end, iii) the cargo-like material is adequate to anchor a molecule of the steric coat to the nanoparticle. In this embodiment, the surface moiety may be conjugated to one end of a linear cargo-like polymer, such as to the amine-terminal end of a homopolymer of an anionic amino acid or an amine-terminated oligonucleotide. A molecule of the steric coat may be conjugated to a moiety incorporated at specific positions within a cargo-like material, such as to thiol or other pendant moiety selectable by an orthogonal chemical conjugation reaction, inserted into an anionic side chain homopolyamide, in one instance by linking together small sections of a homopolyamide with a linker providing said pendant group or in another instance by sequences defined during solid phase synthesis.

In one embodiment, the technology described herein provides for cyclic ROD peptide comprising a lysine conjugated to the carboxyl group of an H₂N-PEG-CO₂H with its distal amine conjugated to the amine of a DNA or RNA oligonucleotide of 10 to 50 bases in length. Such preparations can be used for neovasculature targeted siRNA nanoparticles comprising branched cationic polyamide carrier.

In another embodiment, the technology described herein provides for folic acid conjugated to one end of PEG with its distal end conjugated to the end group thiol formed by thiolysis of an anionic polyacetal such as formed by oxidation of dextran and then coupling of pendant carboxyls via the multitude of aldehyde moieties. Such preparations can be used for the preparation of tumor targeted gene therapy nanoplex comprising gene expression cassette and branched PEI carrier.

In yet another embodiment, the technology described herein provides for a hydrazide moiety conjugated to one end of PEG with its distal end conjugated to the amine terminal end of an anionic polyglutamate coupled to anthrax protective antigen. Such preparations can be used for the preparation of dendritic cell targeted vaccine nanoparticles for prophylactic protection from anthrax infection.

In yet another embodiment, the invention provides for mAb binding peptide linker conjugated to one end of a 2,000 to 10,000 MW PEG with its distal end conjugated to one end of a polyglutamate, or a DNA or RNA oligonucleotide anchor that is from 10 to 50 bases in length. The anchor is a non-functional nucleic acid or polyglutamate, e.g. not cargo. Nanoparticles can be prepared by sequential addition of an aqueous solution of one or more nucleic acid cargo molecules to the above anchor conjugated steric coat and then an aqueous solution of a cationic carrier such as a branched imidazole-amine pendant polyamide cationic macromolecule, and optionally purified by diafiltration with a pharmaceutically acceptable medium.

C. Surface Coat Attachment

In yet another embodiment, the invention provides oriented nanoparticle surface coating via moieties that comprise multiple of sites of attachments to the nanoparticle. The surface coating of the invention provides stability reducing premature cargo release or nanoparticle disassembly, and optionally reduces non-specific nanoparticle interactions. In this embodiment, the surface coating moieties comprise multiple reversible attachments to the nanoparticle, where i) said attachments span two or more nanoparticle components or three or more nanoparticles comoponents, (e.g., carrier), ii) said attachments are reversible permitting cargo release, such as due to changes in biochemical conditions or cleavage within a tissue or upon cell binding, iii) said surface moieties are hydrophilic, preferably not cationic, and iv) said moieties optionally further comprise hydrophilic conjugates and optionally exposed ligands or linkers. In this embodiment, the nanoparticles further comprising a surface decorated material comprising multivalent reversible associations and/or interactions with the nanoparticle, such with carrier or cargo or both. In one instance, the embodiment provides surface coating of nucleic acid nanoparticles comprising polyamide macromolecule carriers with an excess of pendant amines at the surface, coated with a polyacetal, which comprises pendant aldehydes formed from oxidized dextran, by formation of a multitude of Schiff base attachments of the polyacetal with primary amines on the surface. The polyacetal optionally further comprises antibody binding peptide conjugated through a 5000 MW PEG linker via hydrazide binding to a portion of the polyacetal aldehyde moieties. Note that the multitude of associations and/or interactions can be identical or varied, and in one embodiment the associations are largely Schiff base formed between pendant aldehydes and pendant primary amines. The surface associated material may be linear or branched, and in one preferred embodiment the surface associated material exhibits flexibility. In another preferred embodiment the surface associated material is neutral or near neutral in charge or comprises ionic moieties largely zwitterionic. In another embodiment the surface associated material comprises multivalent associations and/or interactions with the surface, and further comprises one or more moieties not directly associated and/or interacting with the surface, such as PEG and exposed ligands. The associations and/or interactions are preferably reversible, and in one preferred embodiment they are cleavable upon tissue uptake and/or cellular internalization.

(i) Multivalent Sulfhydryl Moiety Associations

The technology described herein provides compositions with nanoparticles surface associated material comprising multivalent sulfhydryl moieties. The sulfhydryl associations may be disulfide bonds, thiol-ether bonds, or thiol-inorganic bonds, or other associations, and in a preferred embodiment comprise disulfide bonds. One embodiment provides compositions comprising thiol moieties, which may be provided by the incorporation of cysteine residues. Other embodiments provide compositions comprising cleavable associations and in a preferred embodiment compositions that exhibit release of cargo upon tissue or cellular uptake. Still other embodiments provide compositions comprising cleavable disulfide associations between surface coat and carrier. In one instance, the cleavable compositions forms an amine upon cleavage, one form which employs a dithiol benzyl group that exhibits a reduction mediated release is disclosed by Zalipsky in U.S. Pat. No. 7,238,368.

(ii) Multivalent Schiff Base and Other Amine Associations

The technology disclosed herein also provides compositions with surface associated material comprising multivalent associations with carrier. The associations can be of the form selected from the group of Schiff base, amide, hydrazone, carbamate, and/or amine. These associations may be formed by activated carboxylic acid moiety interaction with amine forming amide, aldehyde interactions with primary amine forming Schiff base, aldehyde interactions with hydrazine forming hydrazone, reduction of Schiff base associations to form an amine, other carbon-nitrogen associations, and their combination. In one embodiment, the molecules of the surface associated material comprises Schiff base formation by aldehyde interactions with primary amines of carriers and optionally are reduced forming a secondary amine. The invention provides compositions comprising aldehyde moieties, and in one preferred embodiment provided by oxidation of carbohydrate moieties. Also provided are compositions comprising cleavable associations, and in some embodiments, compositions that exhibit release upon tissue or cellular uptake.

(iii) Multivalent Ester and Hemi-Acetal Associations

The technology described herein provides compositions with surface associated material comprising multivalent ester associations. These associations may be formed by carboxyl interactions with an alcohol and/or carbohydrate, by aldehyde interaction with alcohols and/or carbohydrates, or other carbon-oxygen associations. In one embodiment, ester formation occurs by carboxyl interactions with alcohol moieties associated with a polyacetal. Another embodiment provides compositions comprising carboxyl moieties, which can be provided by incorporation of amino acids including, but not limited to, aspartic acid and/or glutamic acid moieties. Other embodiments provide compositions comprising cleavable associations, and in some embodiment compositions that exhibit release upon tissue or cellular uptake.

(iv) Linear Compositions

The technology described herein provides compositions with linear materials with multivalent associations. These associations may be formed by moieties throughout the linear composition or may be formed by moieties largely at each end. The compositions may comprise homobifunctional PEG, a linear polyamide, or in one embodiment a linear polyacetal. In one embodiment, the composition comprises PEG with a molecular weight of at least 5,000 Daltons, and in one embodiment an oxidized dextran. In another embodiment, the composition comprises a hydrophilic polypeptide with at least 30% residues selected from the group of serine, threonine, tyrosine, asparagine, glutamine, aspartate, or glutamate.

(v) Branched Compositions

The technology described herein provides compositions with branched materials with multivalent associations. These associations may be formed by moieties throughout the composition or moieties largely at the end of each branch. The compositions may comprise a polyimide, a carbohydrate or in a preferred embodiment a polyacetal. In another embodiment the composition comprises a branched hydrophilic polypeptide with N-terminal threonine residues and with at least 10% residues selected from the group of serine, threonine, aspartamide, glutamide, aspartate, or glutamate.

(vi) Preparation of Compositions

The technology described herein provides for the preparation of compositions with surface materials with multivalent associations. Preparation of such compositions may comprise formation of associations prior to forming the nanoparticles, or in one embodiment by association with a nanoparticles. In the embodiments described herein, unassociated moieties may be quenched by conversion of residual moieties (those not involved in associations) to a different form. In one embodiment residual aldehyde moieties are quenched by formation of Schiff base with an amino-carboxylic acid, the Schiff base may optionally be reduced to an amine. In some embodiments the aldehyde is quenched by reaction with serine, threonine, aspartic acid, and/or glutamic acid.

The technology described herein also provides for preparation of electrostatic nanoparticles by mixing two or more solutions, which upon contact result in solute binding that forms nanoparticles. In one embodiment a preparation is formed by adding one solution into the other while being stirred, and in another embodiment a preparation is formed by injection of the solutions into a static mixer, such as the static mixers used in HPLC instruments for solvent gradients. In both instances the nanoparticles self assemble.

D. Surface Decorated Micellular Drug Nanoparticles

In yet another instance, steric coating conjugates are provided. Steric coated conjugates optionally comprise a ligand or linker, such as cRGD peptide ligand or antibody binding peptide linker, conjugated to one end of a carrier to which cargo can be associated in a manner that the carrier-cargo domain of the conjugate is poorly water soluble. Such instances resulting in formation of micellular nanoparticles comprising a steric surface coating. In forming micellular nanoparticles a carrier is provided that binds unconjugated cargo by reversible or non-covalent bonds, such as conjugates comprising oxidized carbohydrates comprising aldehyde groups that form reversible Schiff base bonds with cargo comprising amine moieties such as doxorubicin, and geldamycin. The conjugates may also comprise unconjugated cargo associated with conjugated cargo, such as doxorubicin precipitates with sulfate ions. In one specific embodiment, an RGD ligand conjugated to one end of a PEG with its distal end conjugated to one end of a polyacetal having a plethora of pendant aldehyde moieties is provided. The pendant aldehyde moieties of the conjugate can be bound to the amine of doxorubicin via a Schiff base, and optionally further associated with unbound doxorubicin via sulfate ions. Such conjugates associated with doxorubicin form a micelle with precipitated doxorubicin in the interior, and an exterior formed from a steric coat comprising exposed RGD ligands. Such micelles are useful for the preparation of neovascualture targeted doxorubicin nanoparticle. In this embodiment, the micellular nanoparticle optionally comprises non-conjugated doxorubicin molecules bound to conjugated doxorubicin via bridging sulfate ions forming a poorly water soluble interior of a micelle. In this embodiment a surface coat conjugate comprising a carrier domain associates with a cargo in a reversible manner, and optionally may further comprise unconjugated cargo, and self associates to form a micellular nanoparticle. In one example a micelle is formed from a cRGD peptide ligand conjugated to one end of a 2,000 to 10,000 MW PEG flexible spacer that is conjugated at the distal end to a polyacetal carrier with pendant aldehyde moieties, such as oxidized dextran, further comprising multiple doxorubicin moieties bound via

Schiff base, and associated into nanoparticles by addition of a sulfate salt, and optionally unconjugated doxorubicin.

### III. Biomedical Applications

A. Ionic Agent Therapeutic Nanoparticle Compositions

In one embodiment, macromolecular compositions are provided that complex ionic agents, and may optionally comprise steric coat optionally conjugated to one or more targeting ligands or linker, for nucleic acid cargo. One embodiment provides a nanoparticle therapeutic formulation of an ionic agent comprising a cationic branched macromolecule optionally associated with an anionic macromolecule and further comprising an integrin targeting ligand peptide coupled through a protective PEG. Conjugates are provided comprising small molecule biologically active agents, such as chemotherapeutics, imaging agents, nutrients etc. Another embodiment provides a nanoparticles comprising one or more cytotoxic agents as cargo and comprising a steric coat comprising cyclic RGD peptide ligands. The conjugate comprises an agent modified to covalently or non-covalently bind doxorubicin, preferably via formation of Schiff base and each conjugate preferably binds more than one doxorubicin. Conjugate coupling can be performed with Boc protection of coupling amines and an orthogonal, e.g., Fmoc, protection of pendant groups. The carboxyl is activated to give the NHS ester and then coupled to unprotected amino groups, and when all coupling is complete all pendant groups deprotected. A carboxyl is activated to give the NHS ester and reacted with the amine moiety of an amine-PEG-carboxyl coupled at its carboxyl to the amine of cRGD-lys, and finally the doxorubicin coupled to the pendant aldehyde moieties of the oxidized disaccharide. In this embodiment, the ROD peptide provides integrin targeting to sites of neovascularization, such as in tumors and eye disease, and the doxorubicin provides anti-proliferative biological activity. The conjugate is optionally incorporated into nanoparticles by self-assembly of doxorubicin by additional of a sulfate salt, such as ammonium sulfate, and optionally unbound doxorubicin. Other embodiments provide, other ligands, and combinations of ligands, such as antibody fragments and/or sialyl Lewis X combined with cyclic RGD, other chemotherapeutic agents such as geldamycin, tubulysin, Velcade, and/or image contrast agents, and combinations thereof. Such embodiments may further comprise branching with pendant moieties to enhance nanoparticle stability and/or intracellular penetration such as TAT peptide of HIV or other protein transduction domains.

B. Imaging Nanoparticle Compositions

In one embodiment, macromolecular compositions are provided that complex imaging agents. One embodiment provides a nanoparticle-formulation of an imaging agent comprising a cationic branched macromolecule optionally associated with an anionic macromolecule and further comprising an integrin targeting ligand peptide coupled through a protective PEG.

C. Vaccine Nanoparticle Compositions

In one embodiment, macromolecular compositions are provided that complex antigens and/or antigen expressing agents and optionally immune stimulating agents or cassettes for their expression. One embodiment provides a nanoparticle vaccine formulation of an ionic antigen comprising a cationic branched macromolecule optionally associated with an anionic macromolecule and further comprising a dendritric cell targeting ligand peptide coupled to the nanoparticle surface.

D. Surface Modified Viral Particles

Macromolecular compositions are provided that modify the surface of viral particles. One composition, shown in FIG. 6C, comprises at least one antibody Fc region binding peptide coupled to a PEG, which is coupled at its distal end to a mixed cationic/hydrophilic branched macromolecule comprising aliphatic end group modifiers. Other examples of surface modified viral particle compositions comprise one or more of; at least one antibody or fragment bound to a macromolecule carrier (in a non-random orientation), and in one embodiment further comprising a reversible or cleavable linkage, and peptide ligand coupled through a PEG linker and 5) at least one additional ligand.

E. Antibiotic Compositions

Macromolecular compositions comprising organic nitrogen pendant groups are provided that exhibit antibiotic activity including antifungal activity. An embodiment, shown in FIG. 6D, comprises mimetics of histatin natural antimicrobial agents. In one embodiment, the invention provides modular elements and the combinatorial construction of a library that can be used with a cell culture drug discovery screen to reveal specific species and embodiments combining 1) potent antimicrobial activity for species and strains included in the cell culture screen and 2) low mammalian cell toxicity for cell types included in the screen.

The technology described herein also provides macromolecular antibiotic compositions that are mimetics of histatin that further comprise one or more of the following: 1) at least one ligand and in one embodiment said ligand is a covalently bound antibody or fragment or analogue (in some embodiments in a non-random orientation), or in another embodiment the ligand is an cyclic ROD peptide; 2) at least one additional ligand with different binding affinity; and 3) at least one hydrophilic polymer, which may comprising a reversible or cleavable linkage.

### IV. Specific Embodiments

A. Histatin Analogue Polyamide Macromolecule Therapeutics:

One embodiment of the technology provide herein is directed to a histatin analogue polyamide macromolecules for the treatment of wounds and microbial infections. In one embodiment, polyamide macromolecules comprise: 4 to 12 branches with arms 10 to 30 amino acids in length comprising 45 to 85% histidine and 15 to 55% lysine content and an oligopeptide core of 3 to 25 amino acids comprising 20 to 100% ornithine, where the core is optionally a circular peptide. Alternatively, the core has ornithines coupled in a dendrimer arrangement with up to 3 generations and optionally beta alanine, serine and/or dPEG₃ (discrete PEG with 3 monomer length) amino acids in between the first and second generation. In another embodiment, polyamide macromolecules are provided with 3 to 25 arms as above coupled to a non-peptide core comprising primary amino moieties, including a branched PEI of up to 5,000 molecular weight, a PAMAM dendrimer with up to 3 generations, a Jeffamine, a branched PEG with up to eight branches terminated in amine moieties, or a polyacetal with pendant primary amine moieties. The macromolecules are produced by a first separate synthesis of arms and core, in a manner giving defined structures or polymerization giving a distribution of structures. In another embodiment, the above macromolecules further comprise 3000 to 10,000 MW PEG conjugates at 5% to 100% of the ends of the arms or optionally a single conjugate at the core and optionally further comprising one or more cRGD peptides appended to the distal end of the PEG. The branched histatin analogue polyamide macromolecules are formulated in a pharmaceutically acceptable manner for topical or parenteral administration, optionally in a lyophilized vial to be reconstituted with water or isotonic saline, for treatment of wounds as a combination wound healing aid and anti-microbial agent and for the treatment of invasive fungal infections.

Benefits over the prior art include improved compositions, utility, and superior methods of production: 1) utility also for wound healing, 2) higher degrees of branching benefiting efficacy, 3) improved localization and penetration of wound and site of infection, and 4) improved synthesis relative to prior art solid phase synthesis of branched histidine-lysine copolymer antimicrobials using a single synthesis sequence starting with the core C-terminus attached to a resin that is more expensive, has lower yields, and is ineffective to produce commercially macromolecules with more than about 4 branches.

B. Antibody Targeted RNA Nanoparticle Reagents:

The technology described herein provides versatile antibody targeted siRNA nanoparticles useful for research. One embodiment comprises nanoparticles formed by mixing an aqueous solution of siRNA containing about 10% fragmented mammalian DNA and further containing 5 to 20% dsDNA oligonucleotides where the DNA is 10 to 30 base pairs in length and conjugated at one end to 5000 MW PEG and where 3 to 25% of the PEG is appended at its distal end to HWRGWV at its N-terminal amine, with an equal volume aqueous solution of cationic carrier such as for in vivo administration low toxicity low molecular weight branched PEI, where the ratio of the carrier weight to nucleic acid weight is within the range from 3 to 7. After incubation for at least 10 minutes, the nanoparticle solution is mixed with a smaller volume, from 1/100^{th} to ⅕^{th}, of an aqueous solution of one or more antibodies and incubated for at least 10 minutes. The resulting solution of siRNA nanoparticles may optionally be purified by diafiltration or dialysis in an electric field. The antibody decorated siRNA nanoparticles may be formulated in a pharmaceutically acceptable manner for administration, optionally in a lyophilized vial to be reconstituted with water or 5% dextrose. To be provided for research, three separate aqueous solutions are provided: polycation carrier, aqueous buffer for siRNA that further comprises HWRGWV-PEG-DNA and aqueous buffer for antibody. Antibodies binding internalized receptors are preferred. The experimentalist provides siRNA and antibodies and prepares the antibody decorated siRNA nanoparticle dispersion for the experiment.

Benefits over prior art include improved compositions, utility, and superior methods of production: 1) utility to decorate siRNA nanoparticle surface with antibody in an oriented fashion for cell and tissue targeted delivery, 2) versatility for selection of carrier, 3) improved efficiency siRNA intracellular release, and 4) better synthesis over prior art.

C. Antibody Targeted RNA Nanoparticle Therapeutics:

The technology described herein provides versatile antibody targeted siRNA nanoparticles useful for research and for therapeutics. In one embodiment, polyamide macromolecules comprising 4 to 12 branches with arms 10 to 30 amino acids in length comprising 45% to 85% amino acids with pendent imidazole appended by a secondary amine via DAP and 15% to 55% ornithine content and an oligopeptide core of 3 to 25 amino acids comprising 20% to 100% ornithine, where the core is optionally a circular peptide or has ornithines coupled in a dendrimer arrangement with up to 3 generations and optionally beta alanine, serine and/or dPEG₃ (discrete PEG with 3 monomer length) amino acids in between the first and second generation. The macromolecules are produced as above for histatin analogues and optionally further comprise PEG and optionally HWRGWV peptide antibody linker conjugated as above. The branched polyamide macromolecules are formulated in a pharmaceutically acceptable manner for parenteral administration with water or 5% dextrose and mixed with an equal volume of an aqueous solution of siRNA to act as cargo at a concentration giving a ratio of polyamide to siRNA in the resulting nanoparticle dispersion from 3 to 7. Note that the siRNA aqueous solution may optionally further comprise 5 to 20% DNA oligonucleotides conjugated to 5 kD PEG with 3 to 25% appended with HWRGWV at the distal end. After incubation for at least 10 minutes, the nanoparticle solution is mixed with a smaller volume, from 1/100^{th} to ⅕^{th}, of an aqueous solution of one or more antibodies and incubated for at least 10 minutes. The resulting solution of siRNA nanoparticles may optionally be purified by diafiltration or dialysis in an electric field. The antibody decorated siRNA nanoparticles may be formulated in a pharmaceutically acceptable manner for parenteral administration, optionally in a lyophilized vial to be reconstituted with water or 5% dextrose. When provided for research, three separate aqueous solutions are provided: polyamide macromolecule, aqueous buffer for siRNA that optionally further comprises HWRGWV-PEG-DNA and aqueous buffer for antibody. Antibodies binding internalized receptors are preferred. The experimentalist provides siRNA and antibodies and prepares the antibody decorated siRNA nanoparticle dispersion for the experiment. When provided for therapeutic applications, vials of antibody decorated siRNA nanoparticle product are manufactured in a production facility and shipped to sites for clinical administration. Benefits over prior art include improved compositions, utility, and superior methods of production: 1) utility to decorate siRNA nanoparticle surface with antibody in an oriented fashion for cell and tissue targeted delivery, 2) higher degrees of polyamide branching, 3) improved efficiency siRNA intracellular release, and 4) better synthesis over prior art as above.

D. cRGD Targeted Nucleic Acid Nanoparticle Reagents and/or Therapeutics:

The technology described herein provides cRGD targeted nanoparticles with cargo comprising siRNA or gene cassettes or both and useful for research and for therapeutics. In one embodiment, polyamide macromolecules are used for antibody decorated siRNA nanoparticles are prepared, also as above, with the exception that the conjugates further comprise cRGD peptide within the same range provided for the antibody binding peptide linker, optionally in addition to the antibody peptide linker. When provided for research, two separate aqueous solutions are provided: polyamide macromolecule and aqueous buffer for siRNA and/or gene cassette that optionally further comprises cRGD-PEG-DNA. siRNA or gene cassettes with biological activity arising from activated endothelial cells and/or integrin expressing tumor cells are preferred. The experimentalist provides siRNA and prepares the cRGD decorated nanoparticle dispersion for the experiment. When provided for therapeutic applications, vials of cRGD decorated nucleic acid nanoparticle product are manufactured in a production facility and shipped to sites for clinical administration. In one form of this embodiment, a cancer therapeutic comprises an unmodified siRNA oligonucleotide inhibitor of one or more angiogenesis pathway genes expressed in neovasculature endothelial cells, such as VEGF R2, VEGF R1, cRaf, and Tie, optionally further comprises a cassette expressing one or more immune stimulating genes, such as GM-CSF and IL-12, and/or genes for secreted anti-tumor proteins, and optionally further comprises anti-Treg antibodies decorated on the nanoparticle surface.

Benefits over prior art include improved compositions, utility, and superior methods of production: 1) utility to decorate nucleic acid nanoparticle surface with cRGD in an oriented fashion for cell and tissue targeted delivery and optionally combine antibody decoration, 2) higher degrees of polyamide branching, 3) improved efficiency nucleic acid intracellular release and optionally provide a combination of gene inhibition and expression, and 4) better synthesis over prior art as above.

E. cRGD Targeted Squalamine Nanoparticle to Treat Angiogenic Diseases

The invention provides cRGD targeted squalamine nanoparticle based therapeutics. Polyamide macromolecules as used for cRGD decorated siRNA nanoparticles are prepared as above. Separately polyamide macromolecules of 10 to 55 amino acids comprising 30% to 100% glutamate and 0 to 40% serine, and optionally comprising 3 to 12 multimers coupled to a polyornithine core in a branch arrangement as above, and optionally further comprising cRGD-PEG conjugates to the N-terminal amine as above, are prepared. Vials of cRGD decorated squalamine nanoparticle product are prepared by mixing an aqueous solutions of squalamine and anionic polyamide followed by incubation for at least 10 min followed by mixing with an aqueous solution of the cationic polyamide followed by incubation for at least 10 min. The resulting nanoparticle dispersion can be purified by diafiltration and may be formulated in a pharmaceutically acceptable manner for parenteral administration, optionally in a lyophilized vial to be reconstituted with water or 5% dextrose.

Benefits over prior art include improved compositions, utility, and superior methods of production: 1) targeting squalamine to sites of angiogenesis, 2) improved pharmacological activity via intracellular release, and 3) inproved synthesis of polyamide components relative to prior art as above.

F. Protective Antigen Targeted Nanoparticle Comprising Antigen for Anthrax Vaccine

In one embodiment, a vaccine to prevent anthrax mediated disease is provided wherein a nanoparticle comprises a cationic branched polyamide carrier and a polyglutamate conjugate of anthrax protective antigen, and optionally further comprising CpG oligonucleotide adjuvant for immune response.

### EXAMPLES

### Example 1

### Antibody Binding Peptide Conjugate: Synthesis of an Antibody Fc Binding Peptide and its Conjugate with PEG

Synthesis is performed of an antibody Fc binding peptide, H(Trt)W(Boc)R(Pbf)G-W(Boc)VA, where all the side chains retained protection groups but the C-terminal carboxyl group is not protected, by solid phase peptide synthesis using the Fmoc chemistry and side chain protecting groups retained by mild cleavage conditions, provided by a commercial custom peptide supplier. The carboxylic acid functional group is used for coupling to an amino-PEG-carboxyl by solution phase DCC mediated coupling.

The protected peptide (50 mg) is dissolved in ethyl acetate (5 ml) and cooled in ice bath. To the above solution, 6.7 mg (1.1 molar equivalent) of DCC (dicyclohexylcarbodiimide) is added and stirred. To the above mixture 3.7 mg of N-hydroxysuccinimide is added and continued stirring for 3 hours. AT the end of 3 hours, the precipitate is filtered off and 100 mg (1 molar equivalent) of NH2-PEG3400-COOH is added and kept for stirring at room temperature for 12 hours. Reaction mixture is filtered to remove the precipitated material and the filtrate is added to petroleum ether to precipitate the material. The precipitate is washed with petroleum ether 3 times and dried.

a) Coupling of Protected Peptide-PEG Conjugate to PEI:

Peptide-PEG conjugate with free a carboxyl group is used to couple to PEI as described above. A solution of peptide-PEG conjugate in DMF is kept in an ice bath and one equivalent of DCC added to the above solution followed by one molar equivalent of N-hydroxy succinimide. The mixture is stirred while in the ice bath for 30 minutes. To this reaction mixture a solution of PEI in DMF is added at a molar ratio of 0.1:1 peptide-PEG conjugate to PEI amine, to conjugate approximately 10% of the PEI amines with peptide-PEG. The reaction mixture is stirred for two hours after the solution gradually warmed to room temperature. The progress of the reaction is monitored using reverse phase HPLC by monitoring the disappearance of peptide-PEG conjugate peak. When the reaction as assessed by this method is substantially complete, the product is precipitated by addition of excess cold ether. The precipitate is washed thoroughly with ether and dried.

b) Deprotection of Peptide in the Peptide-PEG-PEI Conjugate:

The Boo protection groups on the peptide side chain and alpha amino groups are removed by treatment with 50% TFA/dichloromethane for 2 hours at room temperature. The product is precipitated with dry ether and washed with ether 3 or 4 times. The resulting peptide-PEG-PEI conjugate is purified by dialysis using 50,000 MWCO tubing, against 0.1% TFA/Water. The purified material is lyophilized and characterized by NMR to determine the percent conjugation of amines in PEI with PEG-peptide.

c) Synthesis of PEI-PEG-Peptide using SCM-PEG-Mal:

6.1 mg of polyethyleneimine (PEI) of molecular weight 2K is dissolved in 2 ml of 50 mM sodium phosphate adjusted to pH 7.0. To the above solution, 50 mg of SCM-PEG-Mal (3400) (LaysanBio, Arab, Ala.) is added and kept stirring at room temperature for 15 minutes. 27 mg (3 equivalent) of the antibody binding peptide, obtained in the de-protected form with a Cys residue at the C terminal (HWRGWVC) is added to the above reaction mixture and stirred at room temperature for another two hours. The reaction mixture is diluted with 0.05% TFA. The reaction mixture is transferred into 50 KD MWCO (molecular weight cutoff) dialysis tubing and dialyzed extensively for 48 hours with 4 changes of water. The resulting solution is lyophilized and purity checked by RP-HPLC.

An aqueous solution containing the peptide polymer conjugate is adsorbed onto blotting paper or onto the bottom of a 96 well plate. Since the Fc binding peptide is attached to the distal end of the PEG, its exposure for binding an antibody is determined using a labeled (secondary) antibody measured by ELISA.

d) PEI-PEG-Peptide Antibody Binding and Free Peptide Competition:

PPP0 (Pei-PEG-Peptide0) was diluted into the coating buffer (1×TBS, pH7.2) (final concentration: 30 μg/ml) and immediately coated 96-well microplate with 100 □l per well of the diluted PPP0 solution. The plates were sealed and incubated overnight at room temperature.

Wells were aspirated and washed with 300 □l of washing buffer (1×TBS containing 0.1% Tween 20, pH 7.4). The procedure was repeated two more times for a total of three washes

In the next step plates were blocked by adding 300 □l 1% BSA in TBS and incubating for two hours.

Wells were then washed and 50 ul of peptide competitors (antibody binding peptides or unlabeled IgG diluted in binding buffer, using 5-fold serial dilutions starting from 2 mM, total 10-12 points) were added to wells and incubated at room temperature for 15 min. Next, 50 □l of HRP-labeled IgG (˜3 ug/ml in binding buffer) was added to each of the wells. The plate was shaken on a horizontal microplate shaker at 500±50 rpm for 10 rain and then incubate at 37° C. for 2 hours. After incubation, wells were washed 5 times with washing buffer to remove the competitors and HRP-IgG.

150 □l per well of freshly prepared substrate solution was added and incubated for 25-30 min at room temperature. The reaction was stopped by adding 70 □l of 2NH₂SO₄ into each well. Plate was gently tapped to ensure a thorough mixing. Optical density of each well was measured immediately, using a microplate reader set to 450 nm. Data was analyzed GraphPad Prism software.

Formation of PPP/DNA/antibody complexes. PPP0/DNA nanoplexes were formed at a molar ratio of PEI nitrogen to DNA phosphate (N/P ratio) of 6. DNA/PEI polyplexes were prepared by mixing equal volumes of plasmid DNA with PEI in 10 mM HEPES buffer, pH 7.1. Solutions were then briefly vortexed and left for equilibration for 30 min at room temperature. After nanoplexes formed, antibody was added into the nanoplexe solution at a final concentration of 400 □g/ml and incubated for another 30 min.

Purification of PPP-DNA-Ab Nanoparticles from Free Antibodies by Gel Filtration

Sephacryl S-500-HR microspin column was used to purify free antibody and PPP after PPP/DNA-Ab nanoparticle formation. To increase the recovery of nanoparticle, it was diluted in a HST buffer (10 mM HEPES, 140 mM NaCl and 0.01% Tween20). 50-75 DI of nanoparticles are applied to an Sephacryl 5-500 spin column in a antibody mixture and centrifuge at 735 g for 2 min. Gel filtration removed >95% of free antibody from nanoplexes with a high recovery of polyglot components. Up to 80% recovery rates for plasmid DNA were obtained (mean recovery of 53 or 63% for DNA).

Agarose gel electrophoresis. PPP/DNA/Ab NPX. The integrity of PPP0/DNA/Ab nanoparticle was examined by agarose electrophoresis. Heparin was used to release DNA from its complexes at a concentration of 700 □g/ml. Nanoparticle was incubated with or without heparin and were electrophoresed on agarose gels (0.7% w/v, 30 min, 120 V). Intact stayed in the well and heparin released DNA migrated to the gel.

Dot blotting assay. Dot blotting was used to determine antibody binding (before and after purification) to the nanoparticle. Human IgG (400 mg/ml) and PPP0/DNA/IgG nanoparticles (containing 400 mg/ml IgG) were purified from free antibody by Sephacryl S-500-HR microspin column. The purified nanoparticle was spot into a nitrocellular membrane. Anti-human IgG-HRP was used to detect antibody bound to nanoparticle.

### Example 2

### Synthesis of a Branched Cationic Polymer with Pendant Imidazole Moieties Comprising Protective Polymer PEG and Targeting Ligand RGD Peptide

a) Synthesis of a Core Consisting of Ornithine and Ornithine Branch:

Synthesis of the core and branches is carried out by standard solid phase peptide synthesis method. Synthesis starts with peptide synthesis resin coupled with cysteine at low density. Cysteine at the C-terminal will allow conjugation of protective polymer and ligand through the —SH group at the cysteine side chain. To this cysteine, an ornithine is coupled using the alpha and delta amine protected (with Fmoc) derivative of ornithine. This ornithine will act as the core to which additional coupling can be carried out. An ornithine core will provide a branching point allowing coupling to its two amino groups. After the first step of coupling, Fmoc protecting groups is removed by base cleavage. In the next step, another cycle of coupling followed by deprotection is carried out using Fmoc amine protected ornithine. At the end of this cycle, there is four free amino groups available for further reaction. In one embodiment an Fmoc-NH-PEGₙ-NHS is reacted with the four free amino groups, and in another embodiment one more cycle of ornithine coupling and deprotection is performed to give eight free amino groups for further modification followed by a step where Boc-NH-PEGₙ-NHS is reacted with the free amino groups of ornithine to introduce the PEG spacer. Coupling a PEG spacer will reduce the steric hindrance to coupling of the arms to the polymer branches. After this reaction the branched polymer is cleaved from the resin using acidic cleavage, which also will remove the Boc protecting group from the PEG terminus. The branched polymer is purified by HPLC.

b) Synthesis of Ornithine Arms:

The peptide containing the amino acid sequence, (Ornithine)₁₈, is synthesized by solid phase peptide synthesis using the Fmoc chemistry. Rink acid resin or 2-chlorotrityl chloride resin which are amenable to mild acid cleavage of the peptide is used as the solid support for the synthesis of the peptide. Boc protecting group which is stable to the de-protection steps under basic conditions employed for de-protection of Fmoc group, is used for the side chain protection of ornithine. At the final coupling step an ornithine protected with Boc at both alpha and epsilon amino groups is coupled. This fully protected peptide is cleaved from the resin using a cleaving reagent mixture containing 1% trifluoroacetic acid (TFA) in dichloromethane (DCM) as follows. The peptide containing resin is treated with 1% TFA in DCM for 2 minutes and filtered. The filtrate will contain the cleaved but protected peptide. This treatment is repeated 10 times and all the filtrates is collected. The resin is further washed with DCM and methanol. The filtrate and washes is pooled and evaporated to about 5% of the starting volume. To this, water is added to the solution and chilled in ice to precipitate the side chain protected peptide. The precipitated peptide is filtered and dried under vacuum over P₂O₅. The resulting peptide will have a free carboxylic acid group which can be used for coupling to another amine or hydroxyl function. This carboxylic acid functional group is used for coupling of this peptide to the core of the branched peptide to obtain a branched peptide of desired number of branches.

c) Coupling of Protected Ornithine Arm to the Branched Polymer:

Amino groups protected (Ornithine)₁₈ with free carboxyl group is coupled to the free amino groups at the PEG termini. Amine protected (Ornithine)₁₈ with free carboxyl group is dissolved in dry DMF. Three molar equivalents of hydroxybenzotriazole (HOBO dissolved in dry DMF is added to the solution followed by three equivalents of dicyclohexyl carbodiimide (DCC) dissolved in dry DMF. The reaction mixture is stirred at 5 degrees C. for 30 minutes. To this reaction mixture, the branched polymer with free amine is added and stirred for two hours after gradually warming the reaction mixture to room temperature. The resulting polymer conjugate is precipitated by adding a 5 fold excess of cold ether to the DMF solution. The precipitated polymer conjugate is washed several times with ether and purified further by reverse phase HPLC. The polymer is characterized by mass spectral analysis (MALDI) to measure its molecular weight.

The Boc protecting groups on the ornithine arms are removed by treatment with 90% TFA for 2 hours. The resulting peptide is dissolved in water and dialyzed against 0.1% TFA in water. The deprotected polymer is characterized by proton NMR to monitor removal of Boc group.

d) Imidazole Derivatization of Ornithine Arms of the Branched Polymer:

Primary amines of the ornithine arms of the polymer is derivatized to attach pendent imidazole groups by reductive amination using imidazole-2-carboxaldehyde. Reaction between the polymer and imidazole carboxaldehyde mixed in a 1:1 molar ratio of to imidazole to primary amine in the polymer is carried out in 1,2-dichloroethane in presence of a 1.5 molar excess of sodium triacetoxyborohydride. The mixture is stirred at room temperature under nitrogen for two hours. The polymer is precipitated by adding excess cold petroleum ether. The resulting polymer is dialyzed against 0.1% TFA in water. Attachment of imidazole molecule to the primary amines of ornithine by this method of reductive amination will add an ionizable nitrogen to each of the ornithine in the arm thereby increasing the charge density of the arms to enable more effective binding to nucleic acid and other polyanionic molecules.

e) Ligand-Protective Polymer Conjugate:

Linear and cyclic peptides containing RGD sequences is synthesized by standard peptide synthetic methods with its N-terminal amine free. The peptide is coupled to a heterobifunctional PEG, Mal-PEG-SCM through the N-terminal amine. The reaction between the peptide and PEG reagent in 1:1 molar ratio is carried out in dry dimethyl sulfoxide (DMSO) in the presence of one molar equivalent of triethylamine. Progress of the reaction is monitored by reverse phase HPLC. After stirring at room temperature for two hours, the peptide conjugate is precipitated by adding excess cold ether. The precipitate is washed several times with ether and dried. The conjugate is characterized by proton NMR and MALDI.

f) Coupling of Peptide-PEG-Mal to Branched Polymer:

The —SH group of the cysteine residue in the imidazole derivatized branched polymer is used to couple the peptide-PEG conjugate to the polymer. The branched polymer and the peptide-PEG-Mal is mixed together in a 1:1.2 molar ratio and dissolved in DMSO. The pH of the solution is adjusted to 7.5 with triethylamine. The reaction mixture is stirred at room temperature for 3 hours with monitoring by reverse phase HPLC for the progress of the reaction. When the reaction is substantially completed, the polymer is diluted with 0.1% TFA/water and dialyzed extensively using 50,000 MWCO dialysis tubing, to remove unreacted PEG-peptide. The dialyzed polymer is lyophilized and stored.

g) Nanoparticle Formation with Nucleic Acids:

Nanoparticles comprising the branched imidazole pendent polymers and nucleic acid (e.g., plasmid DNA or siRNA) is prepared by self-assembly of the polyanionic nucleic acid moiety with a polycationic branched polymer. An aqueous solution containing nucleic is mixed with a solution containing branched polymer at defined charge ratios to form nanoparticles. Mixing is carried out by combining of the two solutions followed by vortexing, or using a static mixer. Electrostatic interaction between the anionic nucleic acid with the polycation will lead to the formation of particles. The surface protection and colloidal stability of the particle is provided by the PEG surface coat formed during the particle formation. The presence of the surface PEG coat is tested by measuring the surface charge by Zeta potential measurement. The surface PEG coat will reduce the surface charge.

### Example 3

### Synthesis of Branched Cationic Polymer with Polyethyleneimine Core and Cationic Arms Consisting of Pendent Imidazole Groups and Protective Polymer and Targeting Ligand

To synthesize the branched polymer consisting of polyethyleneimine core and imidazole containing arms, polyornithineithine with side chain derivatized with imidazole is coupled to polyethyleneimine as follows.

a) Synthesis of Ornithine Arms:

The peptide containing the amino acid sequence, (Ornithine)₁₈, is synthesized by solid phase peptide synthesis using the Fmoc chemistry. Rink acid resin or 2-chlorotrityl chloride resin which are amenable to mild acid cleavage of the peptide is used as the solid support for synthesis. Boc protecting groups are used to protect amino side chain of ornithine. At the final coupling step an ornithine protected with Boc at both alpha and epsilon amino groups are coupled. This fully protected peptide is cleaved from the resin 1% TFA in DCM as follows. The peptide containing resin is treated with 1% TFA in DCM for 2 minutes and filtered. The filtrate will contain the cleaved but still protected peptide. This treatment is repeated 10 times and all the filtrates are. The resin is further washed with DCM and methanol and the filtrate and pooled washes is evaporated to about 5% of the starting volume. To this solution, water is added and chilled in ice to precipitate the side chain protected peptide with C-terminal carboxylic acid group free. The precipitated peptide is filtered and dried under vacuum over P₂O₅. The resulting peptide will have a free carboxylic acid group which can be used for coupling to another amine or hydroxyl function. This carboxylic acid functional group is used for coupling of this peptide to the core of the branched peptide to obtain a branched peptide of desired number of branches.

b) Coupling of Protected Ornithine Arm to Polyethyleneimine (PEI):

Amino groups protected (Ornithine)₁₈ with free carboxyl group is coupled to the free amino groups of PEI. Amine protected (Ornithine)₁₈ with free carboxyl group is dissolved in dry DMF. Three molar equivalents of hydroxybenzotriazole (HOBt) dissolved in dry DMF is added to the solution followed by three equivalents of dicyclohexyl carbodiimide (DCC) dissolved in dry DMF. The reaction mixture is stirred at 5 degrees for 30 minutes. To this reaction mixture, polyethyleneimine solution in DMF is added and stirred at for two more hours after gradually raising the reaction mixture to room temperature. The resulting polymer conjugate is precipitated by adding a 5 fold excess of cold ether into the DMF solution. The precipitated polymer conjugate is washed several times with ether and purified further by reverse phase HPLC. The polymer is characterized by mass spectral analysis (MALDI) to measure its molecular weight.

The amount of PEI added to the ornithine arm is adjusted to control the number of amines in the PEI derivatized with ornithine arms. Coupling to approximately 10% of the amines of PEI will require a molar ratio of 1:10 of ornithine arm to PEI amine. The percent derivatization can be adjusted by changing the molar ratio of ornithine arms to PEI.

The Boc protecting groups on the ornithine arms are removed by treatment with 90% TFA for 2 hours. The resulting peptide is dissolved in water and dialyzed against 0.1% TFA in water. The deprotected polymer is analyzed by proton NMR to confirm removal of Boc groups.

c) Imidazole Derivatization of Ornithine Arms of the Branched Polymer:

Primary amines of the ornithine arms of the polymer is derivatized to attach pendent imidazole group by reductive amination using imidazole-2-carboxaldehyde. Reaction between the polymer and imidazole carboxaldehyde mixed in a 1:1 molar ratios, of imidazole to primary amines in the polymer is carried out in 1,2-dichloroethane in presence of a 1.5 molar excess of sodium triacetoxyborohydride. The mixture is stirred at room temperature under nitrogen for two hours. The polymer is precipitated by adding excess cold petroleum ether. The resulting polymer is dialyzed against 0.1% TFA in water. Attachment of imidazole molecule to the primary amines of ornithine by this method of reductive amination will add an ionizable nitrogen to each of the ornithine in the arm thereby increasing the charge density of the arms to enable more effective binding to nucleic acid and other polyanionic molecules.

g) Ligand-Protective Polymer Conjugate:

Linear and cyclic peptides containing ROD sequences are synthesized by standard peptide synthetic methods with its N-terminal amine free. The peptide is coupled to a heterobifunctional PEG, VS-PEG-NHS through the N-terminal amine. The reaction between the peptide and PEG reagent in a 1:1 molar ratio is carried out in dry DMSO in the presence of 1 molar equivalent of triethylamine. Progress of the reaction is monitored by reverse phase HPLC. The reaction is ended after it has proceeded substantially to completion. After stirring at room temperature for two hours, the peptide conjugate is precipitated by adding excess cold ether. The precipitate is washed several times with ether and dried. Conjugate is characterized by proton NMR and MALDI.

h) Coupling of Peptide-PEG-VS to Branched Polymer:

Peptide-PEG-VS is coupled to the amino group of PEI. Approximately 10% of the amines is reacted with the Peptide-PEG-VS through coupling of vinyl sulfone groups at pH=9.5. The cationic polymer is dissolved in dry DMF. To this solution a 1:0.1 molar equivalent of PEI amine to Peptide-PEG-VS is added as DMF solution. The pH of the solution is adjusted to 9.5 with triethylamine. The reaction mixture is stirred at room temperature for 24 hours with monitoring by reverse phase HPLC to monitor the progress of the reaction. When the reaction is substantially complete, the polymer is precipitated by adding excess of cold dry ether to the reaction mixture. The precipitate is washed 3 or 4 times with dry ether and dried. The polymer is dissolved in 0.1% TFA/water and dialyzed against 0.1% TFA/water using 50,000 MWCO dialysis tubing. After extensive dialysis, the solution is lyophilized and stored.

g) Nanoparticle Formation with Nucleic Acids:

Nanoparticles comprising the branched imidazole pendent polymers and nucleic acid is prepared by self-assembly of the poly-anionic nucleic acid moiety with a poly-cationic branched polymer. An aqueous solution containing nucleic is mixed with a solution containing branched polymer at defined charge ratios to form nanoparticles. This mixing is carried out by combining the two solutions followed by vortexing, or using a static mixer. Electrostatic interaction between the anionic nucleic acid with the polycation will lead to the formation of particles. The surface protection and colloidal stability of the particle is provided by the PEG surface steric coat formed during particle formation. The presence of the surface PEG coat is tested by measuring the surface charge by Zeta potential measurement. The surface PEG coat will reduce the surface charge to approximately neutral. Biological activity of the nanoparticle is confirmed by cell culture and animal studies involving xenograft tumor models in mice.

### Example 4

### Synthesis of PEI with Polypeptide Arm Consisting of Histidine and Lysine (HK) and Protective Polymer and Targeting Ligand

a) Solid Phase Synthesis of HK Arm of the Branched Peptide:

The peptide containing the amino acid sequence, KHHHKHHHKHHHKHHHK, is synthesized by solid phase peptide synthesis using the Fmoc chemistry. Rink acid resin or 2-chlorotrityl chloride resin both of which are amenable to mild acid cleavage of the peptide is used as the solid support for the synthesis of the peptide. Boc protecting groups which are stable to de-protection steps under basic conditions employed for de-protection of Fmoc group is used for the side chain protection of lysine and histidine amino acids. At the final coupling step a lysine protected with Boc at both alpha and epsilon amino groups are coupled. This fully protected peptide is cleaved from the resin using a cleaving reagent mixture containing 1% TFA in DCM as follows. The peptide containing resin is treated with 1% TFA in DCM for 2 minutes and filtered. The filtrate will contain the cleaved but still protected peptide. This treatment is repeated 10 times and all the filtrates are collected. The resin is further washed with DCM and methanol. The filtrate and washings are pooled and evaporated to about 5% of the starting volume. To this solution, water is added and the solution is chilled in ice to precipitate the side chain protected peptide with C-terminal carboxylic acid groups free. The precipitated peptide is filtered and dried under vacuum over P₂O₅. The resulting peptide will have a free carboxylic acid group which can be used for coupling to another amine or hydroxyl function. This carboxylic acid functional group is used to couple this peptide to PEI.

b) Coupling of amine protected KHHHKHHHKHHHKHHHK to PEI:

The C-terminal carboxyl end of the amine protected KHHHKHHHKHHHHKHHHK peptide is coupled to the amino groups of PEI through DCC/HOBt coupling in DMSO as solvent at 4 degrees C. The HK peptide arm is used at a ratio of 0.1:1 HK peptide arm to the PEI amine, to derivatize about 10% of the amino groups of the PEI. The progress of the reaction is followed by HPLC. The reaction product, which is a branched peptide with PEI core and multiple branches containing side chain protected KHHHKHHHKHHHKHHHK, is precipitated by the addition of cold ether to the reaction mixture. This is further purified by HPLC.

c) Ligand-Protective Polymer Conjugate:

Linear and cyclic peptides containing ROD sequences are synthesized by standard peptide synthetic methods with its N-terminal amine free. The peptide is coupled to a heterobifunctional PEG, VS-PEG-NHS through the N-terminal amine. The reaction between the peptide and PEG reagent in a 1:1 molar ratio is carried out in dry DMSO in the presence of one molar equivalent of triethylamine. Progress of the reaction is monitored by reverse phase HPLC. After stirring at room temperature for two hours, the peptide conjugate is precipitated by adding excess cold ether. The precipitate is washed several times with ether and dried. The conjugate is characterized by proton NMR and MALDI.

d) Coupling of Peptide-PEG-VS to PEI Consisting of Amine Protected HK Arm:

Peptide-PEG-VS is coupled to the amino group of PEI. Approximately 10% of the amines are reacted with the Peptide-PEG-VS through coupling of vinyl sulfone group at pH=9.5. The PEI/HK polymer is dissolved in dry DMF. To this solution a 1:0.1 molar equivalent of Peptide-PEG-VS; PEI amine to Peptide-PEG-VS, is added as DMF solution. The pH of the solution is adjusted to 9.5 with triethylamine. The reaction mixture is stirred at room temperature for 24 hours with monitoring by reverse phase HPLC for the progress of the reaction. When the reaction is completed, the polymer is precipitated by adding excess of cold dry ether into the reaction mixture. The precipitate is washed 3-4 times with dry ether and dried.

Amine protected branched polymer is treated with TFA (90%) to remove the Boc protecting groups from the side chains, to obtain a fully de-protected branched peptide. This polymer is dialyzed against 0.1% TFA/water and lyophilized.

e) Nanoparticle Formation with Nucleic Acids:

Nanoparticles comprising the branched HK polymers and nucleic acid (plasmid DNA or siRNA) are prepared by self-assembly of the poly-anionic nucleic acid moiety with a poly-cationic branched polymer. An aqueous solution containing nucleic is mixed with a solution containing branched polymer at defined charge ratios to form nanoparticles. This mixing is carried out by simple mixing of the two solutions followed by vortexing, or using a static mixer where the two solutions are pumped into a static mixer having a helical mixing element. Electrostatic interaction between the anionic nucleic acid with the polycation will lead to the formation of particles. The surface protection and colloidal stability of the particle is provided by the PEG surface coat formed during the particle formation. The presence of the surface PEG coat is tested by measuring the surface charge by Zeta potential measurement. The surface PEG coat will reduce the surface charge to near neutral. Biological activity of the nanoparticle is confirmed by cell culture and animal studies involving xenograft tumor models in mice.

### Example 5

### Synthesis of Polyornithine with Polypeptide Arm Consisting of Histidine and Lysine (HK) and Protective Polymer and Targeting Ligand

a) Solid Phase Synthesis of HK Arm of the Branched Peptide:

The peptide containing the amino acid sequence, KHHHKHHHKHHHKHHHK, is synthesized by solid phase peptide synthesis using the Fmoc chemistry. Rink acid resin or 2-chlorotrityl chloride resin which are amenable to mild acid cleavage of the peptide is used as the solid support for the synthesis of the peptide Boc protecting groups are used for the side chain protection of lysine and histidine amino acids. At the final coupling step a lysine protected with Boc at both alpha and epsilon amino groups are coupled. This fully protected peptide is cleaved from the resin using a cleaving reagent mixture containing 1% TFA in DCM as follows. The peptide containing resin is treated with 1% TFA in DCM for 2 minutes and filtered. The filtrate will contain the cleaved but protected peptide. This treatment is repeated 10 times and all the filtrates are collected. The resin is further washed with DCM and methanol and all the washings are collected. The filtrate and washes are pooled and evaporated to about 5% of the starting volume. To this, water is added and chilled in ice to precipitate the side chain protected peptide with C-terminal carboxylic acid group free. The precipitated peptide is filtered and dried under vacuum over P₂O₅. The resulting peptide will have a free carboxylic acid group which can be used for coupling to another amine or hydroxyl function. This carboxylic acid functional group is used for coupling of this peptide to PEI.

b) Coupling of Amine Protected KHHHKHHHKHHHKHHHK to poly(Ornithine):

The C-terminal carboxyl end of the amine protected KHHHKHHHKHHHKHHHK peptide is coupled to the amino groups of poly(Ornithine) through DCC/HOBt coupling in DMSO as solvent, at cold temperature. The HK peptide arm is used at a ratio of 0.2:1 HK peptide arm to poly(Ornithine) amine, to derivatize of about 20% of the amino groups of poly(Ornithine). The progress of the reaction is followed by HPLC. The reaction product, which is a branched peptide with poly(Ornithine) core and multiple branches containing side chain protected KHHHKHHHKHHHKHHHK, is precipitated by the addition of cold ether to the reaction mixture. This is further purified by HPLC.

c) Ligand-Protective Polymer Conjugate:

Linear and cyclic peptides containing RGD sequences are synthesized by standard peptide synthetic methods with its N-terminal amine free. The peptide is coupled to a heterobifunctional PEG, VS-PEG-NHS through the N-terminal amine. The reaction between the peptide and PEG reagent in a 1:1 molar ratio is carried out in dry DMSO in the presence of one molar equivalent of triethylamine. Progress of the reaction is monitored by reverse phase HPLC. After stirring at room temperature for two hours, the peptide conjugate is precipitated by adding excess cold ether. The precipitate is washed several times with ether and dried. The conjugate is characterized by proton NMR and MALDI.

d) Coupling of Peptide-PEG-VS to poly(Ornithine) Consisting of Amine Protected HK Arm:

Peptide-PEG-VS is coupled to the amino group of poly(Ornithine). Approximately 10% of the amines are reacted with the Peptide-PEG-VS through coupling of vinyl sulfone group at pH=9.5. The poly(Ornithine)/HK polymer is dissolved in dry DMF. To this solution a 1:0.1 molar equivalent of Peptide-PEG-VS, poly(Ornithine) amine to Peptide-PEG-VS, is added as a DMF solution. The pH of the solution is adjusted to 9.5 with triethylamine. The reaction mixture is stirred at room temperature for 24 hours with monitoring by reverse phase HPLC for the progress of the reaction. Once the reaction is substantially completed, the polymer is precipitated by adding excess of cold dry ether to the reaction mixture. The precipitate is washed 4 times with dry ether and dried.

Amine protected branched polymer is treated with TFA (90%) to remove the Boc protecting groups from the side chain, to obtain a fully de-protected branched peptide. This polymer is dialyzed against 0.1% TFA/water and lyophilized.

e) Nanoparticle Formation with Nucleic Acids:

Nanoparticles comprising the branched HK polymers and nucleic acid is prepared by self-assembly of the poly-anionic nucleic acid moiety with a poly-cationic branched polymer. An aqueous solution containing nucleic is mixed with a solution containing branched polymer at defined charge ratios to form nanoparticles. This mixing is carried out by combining the two solutions followed by vortexing, or by using a static mixer having a helical mixing element. Electrostatic interaction between the anionic nucleic acid with the polycation will lead to the formation of particles. The surface protection and colloidal stability of the particle is provided by the PEG surface coat formed during the particle formation. The presence of the surface PEG coat is tested by measuring the surface charge by Zeta potential measurement. The surface PEG coat will reduce the surface charge to near zero.

### Example 6

### Synthesis of PEI with Protective Polymer and Targeting Ligand and Coating Virus-Like-Particles

a) Synthesis of cRGD-PEG-maleimide

Cyclic peptides containing RGD sequences with an unprotected lysine is synthesized by standard peptide synthetic methods. The peptide is coupled to a heterobifunctional PEG, maleimide-PEG-NHS through reaction of the peptide amine with the NHS end of the PEG. The reaction between the peptide and PEG reagent in a 1:1 molar ratio is carried out in dry DMSO in the presence of one molar equivalent of triethylamine. Progress of the reaction is monitored by reverse phase HPLC. After stirring at room temperature for two hours, the peptide conjugate is precipitated by adding excess cold ether. The precipitate is washed several times with ether and dried. The conjugate is characterized by proton NMR and MALDI.

b) Coupling of Peptide-PEG-maleimide to PEI Consisting of Amine Protected HK Arm:

Peptide-PEG-maleimide is coupled to the amino group of PEI. Approximately 10% of the amines are reacted with the Peptide-PEG-maleimide through coupling of vinyl sulfone group at pH=9.5. The PEI/HK polymer is dissolved in dry DMF. To this solution a 1:0.1 molar equivalent of Peptide-PEG-maleimide; PEI amine to Peptide-PEG-maleimide, is added as a DMF solution. The pH of the solution is adjusted to 9.5 by titrating with triethylamine. The reaction mixture is stirred at room temperature for 24 hours with monitoring by reverse phase HPLC for the progress of the reaction. When the reaction is completed, the polymer is precipitated by adding excess of cold dry ether into the reaction mixture. The precipitate is washed 3-4 times with dry ether and dried.

Amine protected branched polymer is treated with TFA (90%) to remove any Boc protecting groups from the peptide, to obtain a fully de-protected branched peptide. This polymer is dialyzed against 0.1% TFA/water and lyophilized.

c) Nanoparticle Formation with Nucleic Acids:

Nanoparticles comprising the branched HK polymers and nucleic acid (plasmid DNA or siRNA) are prepared by self-assembly of the poly-anionic nucleic acid moiety with a poly-cationic branched polymer. An aqueous solution containing nucleic is mixed with a solution containing branched polymer at defined charge ratios to form nanoparticles. This mixing is carried out by simple mixing of the two solutions followed by vortexing, or using a static mixer having a helical mixing element. Electrostatic interaction between the anionic nucleic acid with the polycation will lead to the formation of particles. The surface protection and colloidal stability of the particle is provided by the PEG surface coat formed by the particle formation. The presence of the surface PEG coat is tested by measuring the surface charge by Zeta potential measurement. The surface PEG coat will reduce the surface charge to near neutral. Biological activity of the nanoparticle is confirmed by cell culture and animal studies involving xenograft tumor models in mice.

### Example 7

### Synthesis of Poly(Ornithine) with Polypeptide Arm Consisting of Histidine and Lysine (HK) and Protective Polymer and Targeting Ligand and Coating Viral Vector Particles for Tissue and Cell Targeting

a) Solid Phase Synthesis of HK Arm of the Branched Peptide:

The peptide containing the amino acid sequence, KHHHKHHHKHHHKHHHK, is synthesized by solid phase peptide synthesis using the Fmoc chemistry. Rink acid resin or 2-chlorotrityl chloride resin which are amenable to mild acid cleavage of the peptide is used as the solid support for the synthesis of the peptide. Boc protecting groups are used for the side chain protection of lysine and histidine. At the final coupling step a lysine protected with Boc at both alpha and epsilon amino groups are coupled. This fully protected peptide is cleaved from the resin using a cleaving reagent mixture containing 1% TFA in DCM as follows. The peptide containing resin is treated with 1% TFA in DCM for 2 minutes and filtered. The filtrate will contain the cleaved but still protected peptide. This treatment is repeated 10 times and all the filtrates are collected. The resin is further washed with DCM and methanol and all the washings are collected collected. The filtrate and washings are pooled and evaporated to about 5% of the starting volume. To this, water is added and chilled in ice to precipitate the side chain protected peptide with C-terminal carboxylic acid group free. The precipitated peptide is filtered and dried under vacuum over P₂O₅. The resulting peptide will have a free carboxylic acid group which can be used for coupling to another amine or hydroxyl function. This carboxylic acid functional group is used for coupling of this peptide to PEI.

b) Coupling of Amine Protected KHHHKHHHKHHHKHHHK to poly(Ornithine):

The C-terminal carboxyl end of the amine protected KHHHKHHHKHHHKHHHK peptide is coupled to the amino groups of poly(Ornithine) through DCC/HOBt coupling in DMSO as solvent, at 4 degrees C. The HK peptide arm is used at a ratio of 0.2:1 HK peptide arms to the poly(Ornithine) amine, to derivatize of about 20% of the amino groups of poly(Ornithine). The progress of the reaction is followed by HPLC methods. The reaction product, which is a branched peptide with poly(Ornithine) core and multiple branches containing side chain protected KHHHKHHHKHHHKHHHK, is precipitated by the addition of cold ether into the reaction mixture. This is further purified by HPLC.

c) Ligand-Protective Polymer Conjugate:

Linear and cyclic peptides containing RGD sequences are synthesized by standard peptide synthetic methods with its N-terminal amine free. The peptide is coupled to a heterobifunctional PEG, VS-PEG-NHS through the N-terminal amine. The reaction between the peptide and PEG reagent in a 1:1 molar ratio is carried out in dry DMSO in the presence of one molar equivalent of triethylamine. Progress of the reaction is monitored by reverse phase HPLC. After stirring at room temperature for two hours, the peptide conjugate is precipitated by adding excess cold ether. The precipitate is washed several times with ether and dried. The conjugate is characterized by proton NMR and MALDI.

d) Coupling of Peptide-PEG-VS to poly(Ornithine) Consisting of Amine Protected HK Arm:

Peptide-PEG-VS is coupled to the amino group of poly(Ornithine). Approximately 10% of the amines are reacted with the Peptide-PEG-VS through coupling of vinyl sulfone group at pH=9.5. The poly(Ornithine)/HK polymer is dissolved in dry DMF. To this solution 1:0.1 molar equivalent of Peptide-PEG-VS, in terms of poly(Ornithine) amine to Peptide-PEG-VS is added as a DMF solution. The pH of the solution is adjusted to 9.5 by titrating with triethylamine. The reaction mixture is stirred at room temperature for 24 hours reverse phase HPLC to monitor the progress of the reaction. When the reaction is completed, the polymer is precipitated by adding excess of cold dry ether to the reaction mixture. The precipitate is washed 4 times with dry ether and dried.

Amine protected branched polymer is treated with TFA (90%) to remove the Boc protecting groups from the side chains, to obtain a fully de-protected branched peptide. This polymer is dialyzed against 0.1% TFA/water and lyophilized.

b) Coating of Viral Vector Particles for Tissue and Cell Targeted Delivery:

The branched cationic polymer with protective polymer and targeting ligand is used to coat the surface of viral vector particles for tissue targeted delivery of vectors for expression of therapeutic genes and also gene inhibitory nucleic acid sequences such as ones expressing antisense nucleic acid sequences, interfering RNA sequences, or micro RNA sequences. The cationic nature of the branched polymer will facilitate its binding to the viral vector surface. The polymers can bind to viral particles made of protein capsids as well as enveloped viruses containing membrane proteins and those devoid of membrane proteins. In case of enveloped viruses, polymers with end modification consisting of hydrophobic moieties that can incorporate into the envelope and provide additional stability is used.

Viral vector particles such as those based on adenovirus, lentivirus, retrovirus, adeno-associated viruses can be coated and targeted to appropriate tissues using the polymer conjugates. Viral vector containing suspensions are combined with different amounts of polymer conjugate solution to determine the amount of polymer needed to completely cover the viral particle surface. Zeta potential measurements are used to confirm the surface coating of the virus with the protective polymer PEG. Chromatographic methods are used to remove the unbound polymer conjugate from the coated viral formulation. Ligands at the end of the PEG will target the coated viral particle to the appropriate tissue and cell, directed by a targeting ligand coupled to PEG.

### Example 8

### Antibody Targeted Nanoparticle and Viral Vector Particles

a) Synthesis of an Antibody Fc Binding Peptide and its Conjugate with PEG:

Synthesis of an antibody Fc binding peptide, HWRGWV, conjugated to PEG is carried out by solid phase peptide synthesis using the Fmoc chemistry. Rink acid resin or 2-chlorotrityl chloride resin which are amenable to mild acid cleavage of the peptide is used as the solid support for the synthesis of the peptide. Resin with an Fmoc-Gly residue is used to couple a heterobifunctional PEG, Fmoc-PEG-SCM, after deprotection of the Fmoc group. Fmoc group at the terminal of PEG is deprotected and the first amino acid (valine) of the sequence is coupled to the amino end of the PEG. Subsequent amino acids according to the sequence is coupled using standard decoupling/coupling cycles using Fmoc chemistry. Boc protecting groups which are stable to de-protection steps under basic conditions employed for de-protection of Fmoc group, is used for the side chain protection of histidines. At the final coupling step a histidine protected with Boc at both alpha amino group and side chain is coupled. This fully protected peptide-PEG conjugate is cleaved from the resin using a cleaving reagent mixture containing 1% TFA in DCM as follows. The peptide containing resin is treated with 1% TFA in DCM for 2 minutes and filtered. The filtrate will contain the cleaved but still protected peptide. This treatment is repeated 10 times and all the filtrates are collected. The resin is further washed with DCM and methanol and all the washings are collected. The filtrate and washings are pooled and evaporated to about 5% of the starting volume. To this, water is added and the solution chilled in ice to precipitate the side chain protected peptide with C-terminal carboxylic acid group free. The precipitated peptide is filtered and dried under vacuum over P₂O₅. The resulting peptide-PEG conjugate will have a free carboxylic acid group which can be used for coupling to another amine or hydroxyl function. This carboxylic acid functional group is used for coupling of this peptide to amino groups of PEI.

b) Coupling of Protected Peptide-PEG Conjugate to PEI:

Peptide-PEG conjugate with free a carboxyl group is used to couple to PEI or other cationic polymers described above. A solution of peptide-PEG conjugate in DMF is kept in an ice bath and one equivalent of DCC is added to the above solution followed by one molar equivalent of HOBt. The mixture is stirred while in the ice bath for 30 minutes. To this reaction mixture a solution of PEI in DMF is added at a molar ratio of 0.1:1:peptide-PEG conjugate to PEI amine, to conjugate approximately 10% of the PEI amines with peptide-PEG. The reaction mixture is stirred for two hours after the solution is gradually warmed to room temperature. The progress of the reaction is monitored using reverse phase HPLC by monitoring the disappearance of peptide-PEG conjugate peak. At the end of the reaction, the product is precipitated by addition of excess cold ether. The precipitate is washed thoroughly with ether and dried.

c) Deprotection of Peptide in the Peptide-PEG-PEI Conjugate:

The Boc protection groups on the peptide side chain and alpha amino groups are removed by treatment with 90% TFA for 2 hours at room temperature. The product is precipitated with dry ether and washed with ether 3 or 4 times. The resulting peptide-PEG-PEI conjugate is purified by dialysis using 50,000 MWCO tubing, against 0.1% TFA/Water. The purified material is lyophilized and characterized by NMR to determine the percent conjugation of amines in PEI with PEG-peptide.

d) Nanoparticle Formation with Nucleic Acids with the Polymer Conjugate:

Nanoparticles comprising the branched polymers and nucleic acid (plasmid DNA or siRNA) is prepared by self-assembly of the poly-anionic nucleic acid moiety with a poly-cationic polymer. An aqueous solution containing nucleic is mixed with a solution containing polymer conjugate at defined charge ratios to form nanoparticles. This mixing is carried out by mixing of the two solutions followed by vortexing, or by using a static mixer having a helical mixing element. Electrostatic interaction between the anionic nucleic acid with the polycation will lead to the formation of particles. The surface protection and colloidal stability of the particle is provided by the PEG surface coat formed by the particle formation. The presence of the surface PEG coat is tested by measuring the surface charge by Zeta potential measurement. The surface PEG coat will reduce the surface charge to approximately neutral. Since the Fc binding peptide is attached to the distal end of the PEG, it is exposed on the surface of the nanoparticle.

e) Binding of Antibody to the Surface of the Nanoparticle:

Nanoparticles with Fc binding peptides on the surface is used to bind antibody molecules that can provide targeted binding of the nanoparticle to selected cells and tissue. Purified monoclonal antibody solutions are added to nanoparticle solution until the surface is saturated with the antibody. Nanoparticle solutions are incubated at 37 degrees in phosphate buffer at pH=7, with varying amounts of antibody solutions to determine the saturation point. The amount of antibody to saturate the nanoparticle is determined by gel electrophoresis analysis of various nanoparticle/antibody mixtures containing different amounts of antibody. Formulations for further studies are prepared at a ratio at which the nanoparticle surface is saturated with antibody molecules. These antibody targeted nanoparticles are evaluated for biological properties using cell culture and animal disease models.

f) Surface Coating of Viral Particles with Antibody Targeted Polymer Conjugates:

Fc binding Peptide-PEG-PEI conjugate is used to coat the surface of viral vector particles. Viral vector particles with protein capsid or enveloped viruses with or without membrane proteins are incubated with the polymer conjugate, in the first step. Electrostatic and hydrophobic interaction between the viral surface and polymer will enable binding of the polymer conjugate to the viral surface. In the second step, coated viral particles are incubated with antibody solutions to enable the binding of the antibody to the peptide, resulting in a viral surface with bound antibody molecules for targeting to desired cells and tissue. These antibody coated viral particles are evaluated in cell culture and disease animal models for their biological properties and delivery of therapeutic agents.

### Example 9

### Immuno-Stimulatory Nanoparticle for Vaccine: Conjugation of Protective Antigen (PA) of Bacillus antharacis with Poly-gamma-D-Glumatic Acid (PGA)

a) Polypeptides

PA is made by expression of a plasmid encoding the PA in E. coli. The expressed protein is purified by chromatographic techniques, for instance by using Q-Sepharose and Superdex-200 columns as described previously in Benson, E L et. al, Biochemistry 37, 3941 (1998) and Rhie, G-E et al, PNAS, 100, 10925 (2003). PGA is synthesized from Bacillus licheniformis or Bacillus subtilis according to the procedures described in Perez-Camero, G. et. al, Biotechnol. Bioeng. 63, 110 (1999) or Kuboto, H et. al, Biosci Biotech Biochem, 57, 1212 (1993), using sonication to reduce its molecular weight.

Conjugation of the PA molecule to PGA is carried out using standard coupling agents, such as water soluble 1-ethyl-3-(3-dimethylaminopropyl) carbodiimide hydrochloride (EDC). EDC couples the PGA carboxylic acid to the amino group of the PA protein through of an amide bond, as described in Rhie, G-E et. al, PNAS, 100, 10925 (2003). Other standard coupling reagents also can be used to prepare this conjugate. The resulting conjugate is purified by column chromatography and the characterized by mass spectrometry.

b): Preparation of Nanoparticles Comprising the PA-PGA Conjugate:

Nanoparticles comprising the PA-PGA conjugate is prepared by the self-assembly of the poly-anionic PGA moiety with a poly-cationic material such as poly-lysine, polyethyleneimine, histidine-lysine co-polymers, or histidine and lysine containing linear or branched peptides. A solution containing PA-PGA conjugate is mixed with a solution containing a polycation at defined charge ratios to form nanoparticles. This mixing is carried out by addition of the solution giving excess charge ratio to the other followed by vortexing, or by using a static mixer having a helical mixing element. Electrostatic interaction between the anionic PGA with the polycation will lead to the formation of particles. The molar ratio of PGA to polycation is adjusted to obtain particles with net negative, neutral or positive surface charge. Particles with net surface charge will provide the colloidal stability to the nanoparticle formulation. To further enhance the stability, surfactants optionally is added to one or both solutions. Samples are prepared with pluronic surfactant. Nanoparticle samples are prepared so that some of the PA molecules are present on the surface of the particle, which will facilitate the uptake of the particle by antigen presenting cells to elicit an immune response against PA and PGA.

c): Chemical Synthesis of Gamma-D-Glutamic Acid Oligomeric Peptides (GDGP) and their Conjugation to PA:

Peptides containing from 10 to 15 consecutive D-Glu residues coupled through the gamma carboxylic acid of the side chain to the alpha amino group of the neighboring C-terminal residue is synthesized by solid phase synthesis using D-glutamic acid derivatives. Three to five amino acid residues of glycine, serine, lysine, alanine or beta-alanine is incorporated into the N-terminus of the peptide to provide a conjugation site through alpha-amine, as well as to provide spacing between the D-glutamic acid block and the conjugated protein. To enable conjugation through sulfhydryl group, a cysteine residue is incorporated at the N-terminus of the peptide. The synthesized peptide is purified by HPLC and characterized by MALDI.

d) Conjugation of Gamma-D-Glutamic Acid Oligopeptide (GDGP) to PA.

D-glutamine peptides containing cysteine at the N terminus is coupled to the Protein (PA) through the sulfhydryl containing side-chain. The protein in aqueous solution of pH between 7 and 8 is reacted with sulfosuccinimidyl-4-(N-maleimidomethyl)cyclohexane-1-carboxylate (Sulfo-SMCC). The maleimide activated protein is purified using a desalting column to remove excess sulfo-SMCC. To the purified protein conjugate, cysteine containing peptide is added and the pH adjusted to between 6.6 and 7.5. The maleimide coupling with the —SH group of the cysteine side chain will yield a PA-peptide conjugate which is further purified by column chromatography. The conjugate is characterized by mass spectrometry to determine the number of peptide molecules coupled to each PA molecule.

A similar coupling procedure is used to couple the N-terminus amine of the peptide to PA amines. In this case, first the peptide is reacted with an excess amount (2-3 molar excess) of a homobifunctional cross linker, disuccinimidyl glutarate (DSG) under anhydrous conditions. The reaction is carried out in dry DMSO or DMF in the presence of 1-2 molar equivalents of base. NHS ester will react with the N-terminal amino group of the peptide. When the reaction is substantially complete, the derivatized peptide is precipitated using dry ether and the precipitate recovered and stored under anhydrous conditions. This peptide with a NHS active terminus is used in a subsequent step to react with PA in aqueous buffer, pH=7 to pH=8, to prepare the conjugate of PA-peptide. This conjugate is purified by column chromatography and characterized by mass spectrometry. The number of peptides conjugated per PA molecule is determined by mass spectrometry.

e) Preparation of Nanoparticles Comprising the PA-GDGP Conjugates:

Nanoparticles comprising the PA-GDGP conjugates are prepared by the self-assembly of the poly-anionic GDGP moiety with a poly-cationic material such as poly-lysine, polyethyleneimine, histidine-lysine co-polymers, or histidine and lysine containing linear or branched peptides. A solution containing PA-GDGP conjugate is mixed with a solution containing a polycation at defined charge ratios to form nanoparticles. This mixing is carried out by addition of the solution giving excess charge ratio to the other followed by vortexing, or using a static mixer having a helical mixing element. Electrostatic interaction between the anionic GDGP with the polycation will lead to the formation of particles. The molar ratio of GDGP to polycation is adjusted to obtain particles with net negative, neutral or positive surface charge. Particles with net surface charge will provide the colloidal stability to the nanoparticle formulation. To further enhance the stability, surfactants optionally is added to one or both solutions. Samples are prepared with pluronic surfactant. Nanoparticle samples are prepared so that some of the PA molecules are present on the surface of the particle, which will facilitate the uptake of the particle by antigen presenting cells to elicit immune response against PA and GDGP.

f): Synthesis of Branched Gamma-D-Glutamic Acid (bGDGP) and its Conjugation to PA:

Branched peptides with two or more arms with each arm consisting of peptides containing 10 and 15 consecutive D-Glu residues coupled through the gamma carboxylic acid of the side chain to the alpha amino group of the neighboring C-terminal reside is synthesized by solid phase synthesis using D-Glu derivatives. Three to five amino acid residues of glycine, alanine or beta-alanine is added to the C-terminus of the sequence to provide spacing between the D-Glu block and the conjugated protein. To enable conjugation through sulfhydryl group, a cysteine residue also may be incorporated at the C-terminus of the peptide. The branching is introduced by incorporation of lysine residues in the sequence N-terminus to the spacer amino acids. Insertion of one lysine in the linear sequence can provide a branching point since further addition of amino acid residues can proceed through both alpha and epsilon amino group of the lysine residue. A second lysine coupling can increase the number of branches to 4. The D-Glu derivatives can then be coupled to the 4 branches simultaneously through the activated gamma carboxylic acid, to generate the branched peptide. The synthesized peptide is purified by HPLC and characterized by MALDI.

g) Conjugation of Gamma-D-Glutamic Acid Oligopeptide (bGDGP) to PA:

bGDGP containing cysteine at the C terminus is coupled to the Protein (PA) through the sulfhydryl side-chain of cysteine and amino groups on the PA. The protein in aqueous solution of pH between 7 and 8 is reacted with sulfosuccinimidyl-4-(N-maleimidomethyl)cyclohexane-1-carboxylate (Sulfo-SMCC). The succinimidyl group will react with amino groups on the protein to form stable amide bonds. The maleimide activated protein is purified using a desalting column to remove excess sulfo-SMCC. To the purified protein conjugate, cysteine containing peptide is added and the pH adjusted to between 6.5 and 7.5. The maleimide coupling with the —SH group of the cysteine side chain will yield a PA-bGDGP conjugate which is further purified using column chromatography. The conjugate is characterized by mass spectrometry to determine the number of peptide molecules coupled to the PA molecule.

h) Preparation of Nanoparticles Comprising the PA-bGDGP Conjugates:

Nanoparticles comprising the PA-bGDGP conjugates are prepared by the self-assembly of the poly-anionic GDGP moiety with a poly-cationic material such as poly-lysine, polyethyleneimine, histidine-lysine co-polymers, or histidine and lysine containing linear or branched peptides. A solution containing PA-GDGP conjugate is mixed with a solution containing a polycation at defined charge ratios to form nanoparticles. This mixing is carried out by addition of one solution to the other followed by vortexing or using a static mixer, having a helical mixing element. Electrostatic interaction between the anionic bGDGP with the polycation will lead to the formation of particles. The molar ratio of bGDGP to polycation is adjusted to obtain particles with net negative, neutral or positive surface charge. Particles with net surface charge will provide the colloidal stability to the nanoparticle formulation. To further enhance the stability, surfactants optionally is added to one or both solutions. Samples are prepared with pluronic surfactant. Nanoparticle samples are prepared so that some of the PA molecules are present on the surface of the particle, which will facilitate the uptake of the particle by antigen presenting cells to elicit immune response against PA and bGDGP.

i) Preparation of Nanoparticles Comprising HK Polymer and PGA Polymer:

Nanoparticles comprising HK polypeptide polymers and PGA is prepared by self-assembly of the poly-anionic PGA moiety with a poly-cationic histidine-lysine co-polymers of linear or branched form. A solution containing PGA is mixed with a solution containing HK polymer at defined charge ratios to form nanoparticles. This mixing is carried out by addition of the solution giving excess charge ratio to the other followed by vortexing, or using a static mixer having a helical mixing element. Electrostatic interaction between the anionic PGA with the polycation will lead to the formation of particles. The molar ratio of PGA to polycation is adjusted to obtain particles with net negative, neutral or positive surface charge. Particles with net surface charge will provide the colloidal stability to the nanoparticle formulation. To further enhance the stability, surfactants optionally is added to one or both solutions. Samples are prepared with pluronic surfactant. Nanoparticle samples are prepared so that some of the PGA molecules are present on the surface of the particle, which will protect the nanoparticle from phagocytic cell uptake.

### Example 10

### RGD Targeted Nucleic Acid Carrying Nanoparticle Using Pendant Imidazole-Amine Branched Polyamide and Internal Scaffold

a) Solid Phase Synthesis of Pendant Imidazole-Amine Arm:

The polyamide arm containing a defined sequence of imidazole (Im) and amine (Am) pendant groups, AmImImImAmImImImAmImImImAmImIMImAm, is synthesized by solid phase synthesis to yield a polyamide with the imidazole and amine moieties fully protected with Boc and a free carboxyl at the C-terminal end of the polyamide using several non-natural amino acids: Fmoc-N-(Boc-Im)-N′-diaminoproprionic acid [for Im units in the sequence], Fmoc-N-(Boc)-N′-diaminoproprionic acid [for the Am units in the sequence except N-terminal], and Boc-N-(Boc)-N′-diaminoproprionic acid [for the N-terminal Am]. The Fmoc-N-(Boc-Im)-N′-diaminoproprionic acid monomer is prepared by coupling imidazole aldehyde to Fmoc-N-diaminoproprionic acid at neutral pH and reducing the Schiff base with sodium borohydride, and then reacting the imidazole to form Boc protected imidazole. The polyamide will have a free carboxylic acid group used for coupling to a core with amine or other function.

b) Linear Polyamine Core with N-Terminal Threonine:

A linear core polyamide with pendant amines (Am) and serine (S) or threonine (T) hydroxyls are synthesized by solid phase with the sequence TAmTAmTAmTAmTAmS using Fmoc chemistry and cleavage so that pendant amities remain Boc protected. The synthesis will use natural and non-natural amino acids: Fmoc-Threonine, Fmoc-Serine, and Fmoc-N-(Boc)-N′-diaminoproprionic acid [for Am units in the sequence]. This is purified by HPLC.

c) Pendant Imidazole-Amine Branched Polyamide Carrier

The core is deprotected by cleaving the Boc groups from the amines. Boc protected material is treated with TFA (90%) to remove the Boc protecting groups, to obtain a fully de-protected material. Then the material is dialyzed against 0.1% TFA/water and lyophilized. Then the C-terminal carboxyl end of the imidazole and amine protected arm is coupled to core amino groups using DCC/HOBt coupling as above. An arm to core amine ratio of 2:1 is used to derivatize of a majority of the amino groups of the core. The reaction progress is monitored by HPLC to yield a branched polyamide with 4-6 arms coupled to the core and the product precipitated by addition of cold ether. The product is deprotected by treatment as above and the final carrier is purified by HPLC as the TFA salt.

d) Peg Conjugate to a Oligodeoxyribonucleic Acid Moiety

A linear single stranded DNA 21mer oligonucleotide scaffold is prepared by solid phase with a sequence of AAUAAUAAUAAUAAUAAUAAU and is modified to have a 5′ terminal amine. A hydrazide-PEG conjugate of the oligonucleotide is prepared by reaction of the 5′ amine with a Boc-hydrazide-PEG-NHS, in cold DMSO and then the Boc protecting group removed as described, producing Hz-PEG-DNA.

e) RGD Targeted Nanoparticles for Neovasculature Targeted Delivery:

An siRNA oligonucleotide is dissolved at 1 mg/ml in a distilled water solution of Hz-PEG-DNA at 0.05 to 0.5 mg/ml. Then the solution is added drop-wise to an equal volume of an aqueous solution of carrier at 2-6 mg/ml in water in a 15 ml tube while being vortexed, and the resulting nucleic acid carrying nanoparticles are allowed to stand at room temperature for at least 30 minutes. Separately, a cyclic RGD peptide with a lysine epsilon amine is converted to an aldehyde in an aqueous buffer at pH 7. A 10× aqueous solution of the aldehyde containing cRGD is added to the nanoparticle solution to give a final concentration of 5 to 200 microgram/ml and is allowed to stand at room temperature for at least 30 min. The resulting solution is dialyzed against 100× volume of 5 mM HEPES at pH=7.5 for 1 hr and then against 100× volume of 5% glucose dissolved in 5 mM HEPES at pH=7.5 for 1 hr. The resulting RGD targeted nanoparticles are stored at 4 degrees C.

### Example 11

### Nanoparticle Formulations with Anti-Proliferate Molecules

a) Preparation of Nanoparticles Comprising Squalamine and PGA:

Nanoparticles comprising cationic squalamine is prepared by self assembly of squalamine poly-anionic GDGP, bGDGP or PGA. A solution containing squalamine is mixed with a solution containing a polyanion at defined charge ratios to form nanoparticles. This mixing is carried out by addition of one solution to the other followed by vortexing or using a static mixer, having a helical mixing element. Electrostatic interaction between the anionic glutamic acid with the cationic squalamine will lead to the formation of particles. The molar ratio of polyanionic species to squalamine is adjusted to obtain particles with net negative, neutral or positive surface charge. Particles with net surface charge will provide the colloidal stability to the nanoparticle formulation. To further enhance the stability, surfactants or hydrophilic polymers such as PEG is incorporated into the nanoparticle through covalent bonding or by non-covalent interaction.

b) Preparation of Nanoparticle Comprising Squalamine Conjugated to Cationic Polypeptide.

The amino group of squalamine is conjugated to cationic polypeptides consisting lysine, or histidine and lysine through using homobifunctional cross linkers described in Example 3, or through coupling with a dithiol benzyl that exhibits reduction mediated release of squalamine as described in Zalipsky et al. U.S. Pat. No. 7,238,368.

The squalamine-polycation conjugate is mixed with PGA, GDGP, or bGDGP to form nanoparticles. This mixing is carried out by addition of one solution to the other followed by vortexing or using a static mixer, having a helical mixing. Electrostatic interaction between the anionic glutamic acid with the cationic squalamine-polycation conjugate will lead to the formation of particles. The molar ratio of polyanionic species to squalamine-polycation conjugate is adjusted to obtain particles with net negative, neutral or positive surface charge. Particles with net surface charge will provide colloidal stability to the nanoparticle formulation. To further enhance stability, surfactants or hydrophilic polymers such as PEG is incorporated into the nanoparticle through covalent bonding or by non-covalent interaction.

### Example 12

### Preparation of Polyacetal with Pendant Aldehyde and Alcohol Moieties

Polyhydroxymethylacetal-aldehyde (PHAA) is prepared via lateral cleavage of carbohydrate rings by periodate oxidation. Dextran, from 9-70 kDa (0.162 g/mmol by glucopyranoside) is dissolved at 0.051 g/mL in deionized water. Dextran solution at 0-5° C. is mixed with sodium metaperiodate at 0.2 to 1.1 mole equivalent (0.214 g/mmol) dissolved in deionized water at 0.14 g/ml at 0-5° C. in a light-protected glass reactor and incubated for 3 h. The precipitated sodium iodate is removed by filtering the reaction mixture (glass filter). The pH of the filtrate is adjusted to 7.0 with 1 N NaOH. The obtained macromolecular product is desalted and concentrated by dialysis or on a Centricon dialysis system (Amicon, Beverly, Mass.) equipped with a hollow fiber cartridge, cutoff 30 kDa, by passing approximately 6 volumes of deionized water through the polymer solution. Alternatively, the product can be purified on a Sephadex G-25 preparative column using deionized water as an eluent. PHAA is recovered from aqueous solutions by lyophilization.

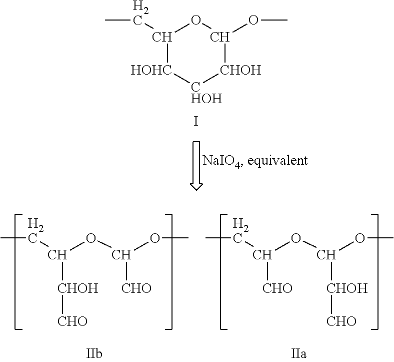

For optional partial reduction of pendant aldehydes to pendant alcohol moieties, the pH of the filtrate is adjusted to 8.0 with 5 N NaOH, and the resultant solution treated with sodium borohydride (0.037 g/mmol) at 0.1 to 1 mole equivalent of periodate treatment dissolved in deionized water at 0.074 g/ml for 2 h at 0-5° C. Then, the pH is adjusted to approximately 7.0 with 1 N HCl. The obtained macromolecular product is desalted, concentrated, and lyophilized as above.

### Example 13

### Preparation of Cationic Nucleic Acid Nanoparticle

Cationic nucleic acid nanoparticle comprising branched cationic polyamide is prepared by self-assembly of the poly-anionic nucleic acid with an excess of poly-cationic imidazole-amine containing branched polyamide. An aqueous solution containing nucleic acid is mixed with a solution containing aqueous polycation at defined charge ratios of 1:1.5 to 1:10 nucleic acid:polycation to form nanoparticles. This mixing is carried out by simple addition of the solution encountering excess charge ratio (nucleic acid) to the other followed by vortex, or using a static mixer where the two solutions are pumped into a static mixer to be mixed within the helical mixing element of a static mixer. Electrostatic interaction between the anionic nucleic acid with the polycation will lead to the formation of particles. The molar ratio of nucleic acid to polycation is adjusted to obtain particles with positive surface charge, in one instance the ratio is 1:4 nucleic acid:polycation by weight. Particles with net surface charge will provide some colloidal stability to the nanoparticle formulation. In order to further enhance the stability, a non-ionic surfactant optionally is added to one or both solutions before mixing, at a concentration approximately equal to the surfactant CMC prior to mixing. Surfactant is prepared with Tween, E8Cl2, octaglucoside, pluronic or Brij. Nanoparticles are dialyzed and stored at 5° C.

### Example 14

### Preparation of PHAA Stabilized Nucleic Acid Nanoparticles

Nucleic acid nanoparticles comprising HK polypeptide (Example 2) in low salt aqueous solution is treated with PHAA at a molar equivalent of 2-100 of exposed primary amine at 0-5° C. for 2 hr. For example, 1 mg/ml of 1:4 wt ratio nucleic acid:HK polypeptide with 25% residues with primary amine nanoparticle is estimated as equivalent to 0.02 mg/ml or 0.0002 mmol/ml exposed primary amine resulting in addition of equal volume of PHAA at 0.032-0.16 mg/ml. The obtained product is desalted and concentrated by dialysis or on a Centricon system (Amicon, Beverly, Mass.). For optional reduction of Schiff base to amine, the pH is adjusted to 7.5, and the resultant solution treated with sodium borohydride (0.037 g/mmol) at 2 mole equivalent of calculated aldehyde content dissolved in deionized water at 0.074 g/ml for 2 h at 0-5° C. Then, the pH is adjusted to approximately 7.0 with 1 N HCl. The product is dialyzed and concentrated as above (Example 1).

### Example 15

### Preparation of Materials Comprising Pendant Aldehyde End Group Moieties

Homobifunctional PEG with terminal aldehyde moieties are prepared by first preparing PEG with carbohydrate moieties at both ends (dicarbohydrate-PEG or DC-PEG) using chemical reactions employed in solid phase peptide synthesis as follows. Diamino-PEG in DMF or other polar non-aquoeus solvent is mixed with 3 equivalents of glucuronic acid and reacted to form amide bonds with the PEG end groups with DCC or other peptide coupling reagent using conditions typical of solid phase synthesis. The product is recovered by dilution with deionized water, filtration, dialysis, and finally lyophilized. Optionally di-carboxyl-PEG is reacted with glucosamine or galactosamine using similar reaction conditions and recovery of product. Di-aldehyde-PEG (PEG-DA) is prepared via lateral cleavage of the carbohydrate end groups by periodate oxidation. DC-PEG, prepared with 1-20 kDa homobifunctional PEG, is dissolved at 0.05 g/mL, carbohydrate in deionized water, taking into account the weight of only the carbohydrate end groups. For PEG of 1 kDa total DC-PEG is 0.2 g/ml, for 2 kDa total is 0.36 g/ml, for 10 kDa total is 1.6 g/ml. PEG-DC solution at 0-5° C. is mixed with sodium metaperiodate (0.214 g/mmol) at 1.1 mole equivalent to carbohydrate dissolved in deionized water at 0.15 g/ml at 0-5° C. in a light-protected glass reactor and incubated for 3 b. The product is desalted, concentrated, and lyophilized as above (Example 1).

Hydrophilic polypeptides with terminal aldehyde moieties are prepared by first preparation of N-terminal threonine peptide branches by solid phase synthesis either on a multivalent amine core such as tri-lysine by solid phase synthesis or by post-synthesis coupling to a core such as Jeffamine. Hydrophilic polypeptides are prepared with at least 30% residues selected from the group of serine, threonine, aspartamide, glutamide, aspartate, or glutamate. Branched polypeptides with N-terminal aldehyde moieties are prepared via oxidation of the N-terminal Threonine residue end groups by periodate, using reaction conditions as above.

### Example 16

### Preparation of Stabilized Nanoparticle with Targeting Ligand-PEG Conjugate

Surface exposed targeting ligand and surface PEG conjugated stabilized nanoparticles are prepared by coupling ligand, PEG, or ligand-PEG moieties to stabilized nanoparticles. In this example, the coupling is with surface aldehyde moieties. First, ligand, PEG, or ligand-PEG moieties are prepared with a terminal amine moiety, and in this example a single amine moiety, for coupling to exposed aldehyde moieties on the surface of stabilized nanoparticle preparations. A cyclic RGD peptide is commercially available with a Lys residue with a free amino moiety (Peptides International), and can be used directly or to couple to an amino-protected omega-amino-PEG-carboxyl reagent, also commercially available, using peptide synthesis coupling chemistry as above (Example 4) followed by deprotection of the omega-amino-PEG. For coupling PEG without ligand methoxy-PEG-amine is commercially available.

Stabilized nanoparticles are prepared with multivalent pendant aldehyde groups, for example as in Example 3, but optional reduction will not be performed. Then the resulting nanoparticles are mixed with a low salt aqueous solution of the ligand, PEG, or ligand-PEG with a free amine moiety at 0-5° C. in a light-protected glass reactor and incubated for 3 hr. For optional reduction of Schiff base to amine, the pH is adjusted to 7.5, and the resultant solution treated with sodium borohydride (0.037 g/mmol) at 2 mole equivalent of calculated aldehyde content dissolved in deionized water at 0.074 g/ml for 2 h at 0-5° C. Then, the pH is adjusted to approximately 7.0 with 1 N HCl. The product is dialyzed and concentrated as above (Example 1).

Surface protection and colloidal stability of the particle is enhanced by the PEG surface coat. Presence of the surface PEG coat is tested by measuring the surface charge by Zeta potential measurement. Presence of the surface PEG coat will reduce the surface charge to near neutral. Since the cRGD peptide is attached to the distal end of the PEG, it is exposed on the surface.

Nanoparticle with Fc binding peptide on the surface is prepared using H₂N-HWRGWV-CO₂H, instead of cRGD peptide, to bind antibody molecules that can provide targeted binding of the nanoparticle to selected cells and tissue. Purified monoclonal antibody solutions are added to nanoparticle solution until the surface is saturated with the antibody. Nanoparticle solutions are incubated at 37 degree in phosphate buffer at pH 7, with varying amounts of antibody solutions to determine the saturation point. The amount of antibody to saturate the nanoparticle is determined by gel electrophoresis analysis of various nanoparticle/antibody mixtures containing different amounts of antibody. Formulations for further studies are prepared at ratio at which the nanoparticle surface is saturated with antibody molecules. This antibody targeted nanoparticle will be evaluated for biological properties using cell culture and animal disease models.

### Example 17

### Synthesis of PEI-PEG-c(RGDfK) using SCM-PEG-Mal

Cyclic RGD peptide c(RGDfK) is obtained from Peptides International. 10 mg of the RGD peptide is dissolved in 2 ml dry DMF. To this 64 mg (1.1 eq) of SCM-PEG-Mal (3400 mol wt) is added followed by 3 ul of N,N-diisopropylethylamine (DIPEA). The reaction mixture is kept stirring at room temperature for 3 hours. After 3 hours of stirring, 7.2 mg of PEI (2K) is added to the above solution. 4.5 ul more of DIPEA is added and the reaction mixture is kept stirring for 24 hours. Progress of the reaction is monitored by HPLC. The conjugate is precipitated by adding the reaction mixture into cold ether. The precipate is collected and washed with ether 3 times and dried.

The sample is dissolved in 0.05% TFA/water and transferred into a 50 Kd MWCO dialysis tubing and dialyzed extensively against 0.05% TFA/water for 48 hours. The solution was then lyophilized to obtain solid material.

One skilled in the art will recognize that the invention is not limited to the descriptions and embodiments herein.

All references recited herein are incorporated by reference.

### Example 18

### Antibody Decoration of Liposome Nanoparticle

a) Synthesis of Lipid-PEG-P0 Conjugate:

The antibody binding peptide with an additional Cys at the terminus will be coupled to a lipid-PEG conjugate with a reactive maleimide moiety (1,2-Distearoyl-sn-Glycero-3-Phosphoethanolamine-PEG-Mal; DSPE-PEG-Mal). 100 mg of DSPE-PEG-Mal (Avanti Polar Lipids Inc.) will be dissolved in 5 ml of dry DMF (N,N-dimethyl formamide) and 70 mg of peptide (HWRGWVC) will be added to the above solution and stirred at room temperature for 3 hours. The progress of the reaction will be followed by TLC. At the end of the reaction, dry ether will be added to precipitate out the material. The conjugate will be further purified by RP-HPLC and characterized by NMR and Mass Spectrometry.

b) Preparation of Liposome Comprising Surface Exposed PEG-P0 Conjugate:

DSPE-PEG-P0 conjugate will be incorporated into cationic liposome for nucleic acid delivery as follows. A mixture of 1,2-Dioleoyl-3-trimethyl ammonium propane (DOTAP), Cholesterol and DSPE-PEG-P0 will be dissolved in dry Chloroform in 5:4:1 molar ratio. The chloroform solution of the mixed lipids will be slowly evaporated in a glass tube into a thin film using a rotary evaporator. The lipid mixture will be suspended in 10 mM HEPES buffer, pH 7.3 and vortexed vigorously. The liposome suspension will be extruded through 100 nm polycarbonate membrane 5 times, to reduce the size of the liposome to an average 100 nm size.

c) Preparation of Nucleic Acid-Liposome Nanoparticle with Surface Exposed PEG-P0 and then Antibody Decoration.

Equal volumes of DOTAP/Chol/DSPE-PEG-P0 liposome in HEPES buffer and nucleic acid in HEPES buffer will be mixed by rapid addition of the nucleic acid solution into the liposome solution followed vigorous vortexing for 30 seconds. The concentration of the two solutions will adjusted to obtain nanoparticle compositions of varying N/P (amino function of DOTAP/phosphate of nucleic acid) ratios. Into the lipid-NA complex suspension, antibody solution will be added and incubated at 37 degree for 2 hours before use. Optionally unbound antibody can be removed by dialysis of gel filtration.

1. A nanoparticle comprising;
a. a cargo,
b. a carrier which binds to the cargo wherein said carrier comprises a core having bound thereto from two to ten polypeptide arms;
wherein one or more of the arms is optionally branched; and
wherein the arms are comprised of amino acid monomers where 30% to 100% of the amino acid monomers comprise pendant anion or cation groups that are charged at neutral pH, provided that at least 75% of said pendent groups on said carrier have the same sign of charge;
provided that less than 5% of said amino acid monomers are histidine and less than 5% of said amino acid monomers are lysine.

2. The nanoparticles of claim 2, wherein the amino acid monomers comprise pendant cation groups.

3. The nanoparticles according to claim 2, wherein the pendant cation groups comprise one or more groups selected imidazole rings and primary amine groups.

4. The nanoparticles of claim 2, wherein 50% to 100% of the amino acid monomers comprise pendant cation groups

5-25. (canceled)

26. The nanoparticle of claim 1 wherein the cargo is selected from the group consisting of a therapeutic agent, a diagnostic imaging agent, and an experimental compound.

27. A polyimide comprising a core having bound thereto from two to ten polypeptide arms; wherein one or more of the arms is optionally branched; and wherein the arms comprise amino acid monomers where 50% to 100% of the amino acid monomers comprise pendant cation groups that are charged at neutral pH; with the proviso that the amino acid monomers do not include L-histidine or L-lysine.

28-31. (canceled)

32. A method for treating a fungal infection in a patient comprising administering to the patient a polyamide of claim 27, in a pharmaceutically acceptable formulation.

33-35. (canceled)

36. A nanoparticle comprising;
a. a cargo,
b. a universal-carrier which binds to the cargo,
c. an anchor, which is bound to a molecule of a steric coat by a linker, wherein said anchor associates with said cargo or said carrier.

37. The nanoparticle of claim 36, wherein said molecule of the steric coat further comprises a second linker, a ligand, or both a second linker and a ligand, attached to said molecule at a location distal to the anchor.

38. The nanoparticle of claim 37, wherein said location distal to the anchor is the distal terminus of said steric coat molecule.

39. The nanoparticle of claim 37, wherein the linker comprises a non-covalent antibody binding moiety wherein said moiety does not bind to the antigen binding site of said antibody.

40. The nanoparticle of claim 39, wherein the non-covalent antibody binding moiety comprises a peptide selected from the group consisting of HWRGWVC (SEQ ID NO.: 7), HWRAWA (SEQ ID NO.: 8), HWRGWA (SEQ ID NO.: 9). HWRGWL (SEQ ID NO.: 10), HWRAWV (SEQ ID NO.: 11), HFRRHL (SEQ ID NO.: 4), HFRRHI (SEQ ID NO.: 12), HFRRHA (SEQ ID NO.: 13), HVHYYW (SEQ ID NO.: 5), HAHYYW (SEQ ID NO. 14), YYWLHH (SEQ ID NO.: 6), and HYFKFD (SEQ ID NO.: 3).

41. The nanoparticle of claim 39, further comprising one or more antibodies, or fragments thereof, independently selected from the group consisting of: polyclonal antibodies, monoclonal antibodies, single chain antibodies Fc fragments and Fab fragments.

42. The nanoparticle of claim 39, further comprising one or more antibodies, or fragments thereof, independently selected from the group consisting of IgA, IgB, IgD, IgE, IgG, IgH and IgM.

43. The nanoparticle of claim 37, wherein the ligand is selected from polypeptides, receptor binding proteins, folate, carbohydrates such as sialyl Lewis X, apoB, EGF, VEGF, FGF, tenascin, RGD, and cyclic RGD.

44-46. (canceled)

47. The nanoparticle of claim 36, wherein said cargo is selected from the group consisting of nucleic acids, polypeptides, proteins, antibodies, phosphorylated compounds, therapeutic agents, and imaging agents.

48. The nanoparticle of claim 47 wherein said cargo is a phosphorylated compound selected from the group consisting of nucleotides, phosphopeptides, phosphoproteins, and organophosphorus compounds.

49. The nanoparticle of claim 36, wherein said anchor comprises a polyelectrolyte.

50. The nanoparticle of claim 49 wherein said polyelectrolyte is a polyamide with charged pendent groups.

51. The nanoparticle of claim 49 wherein said polyelectrolyte is a nucleic acid.

53-54. (canceled)
